(19) United States
(12) Patent Application Publication
(10) Pub. No.: US 2022/0380476 A1
(43) Pub. Date: Dec. 1, 2022

(54) Use of an anti-CCR7 antibody in combination therapies with a BTK inhibitor and/or BCL2- inhibitor for treating hematological malignancies

(71) Applicants: Catapult Therapeutics B.V., Lelystad (NL); Immunological and Medicinal Products, SL, Madrid (ES)

(72) Inventors: Carlos Cuesta Mateos, Madrid (ES); Tamara Mateu Albero, Ontinyent Valencia (ES); Cecilia Muñoz Calleja, Algete (ES); Fernando Terrón Fernández, Madrid (ES)

(73) Assignees: Catapult Therapeutics B.V., Lelystad (NL); Immunological and Medicinal Products, SL, Madrid (ES)

(21) Appl. No.: 17/767,096

(22) PCT Filed: Oct. 9, 2020
(86) PCT No.: PCT/EP2020/078359

§ 371 Date: Apr. 7, 2022

(30) Foreign Application Priority Data
Oct. 9, 2019 (EP) 19382883.7

(51) Int. Cl. C07K 16/28; A61K 31/519; A61K 31/635; A61K 31/495

(52) U.S. Cl. CPC C07K 16/2866; A61K 31/519; A61K 31/635; A61K 31/495; C07K 2317/20; C07K 2317/76; C07K 2317/734; C07K 2317/732

(57) ABSTRACT

The present invention provides a novel use and methods comprising antibodies, or antigen-binding fragments thereof, which bind to a CCR7 receptor for use as a novel combination therapy with a BTK inhibitor and/or a Bcl-2 inhibitor in treatment of hyperproliferative blood malignancies, preferably in B-cell lymphomas, such as CLL. The combination can be used as first line, or in naïve patients not treated before with a BTK inhibitor and/or Bcl-2 inhibitor, or in patients with a BTK-inhibitor and/or Bcl-2-inhibitor refractory/relapsed disease. The antibodies and antigen-binding fragments are capable of selectively depleting ex vivo or in vitro malignant cells expressing CCR7 and are capable of impairing/blocking migration of said tumor cells towards CCR7 ligands. These effects are not related to previous or contemporary treatments with a BTK inhibitor and/or a Bcl-2 inhibitor. Similarly, the efficacy of the antibodies is not affected in patients that have relapsed/refractory disease. The use of said antibodies as a monotherapy or as a combination with a BTK inhibitor and/or a Bcl-2 inhibitor for depleting, killing and impairing/blocking migration and activation of tumor cells expressing CCR7 cells is disclosed, thus providing an alternative therapy treating hyperproliferative blood cancers.

### FIELD OF THE INVENTION

The present invention relates in general to the fields of medicine and pharmacy, in particular to the field of oncology.

### BACKGROUND ART

Effective treatment of hyperproliferative disorders is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis as a result of such deregulation, which can includes abnormalities in signal transduction pathways.

Bruton's tyrosine kinase (BTK), a member of the Tec family of cytoplasmic tyrosine kinases, is intimately involved in multiple signal-transduction pathways regulating survival, activation, proliferation, and differentiation of B-lineage lymphoid cells. BTK is an upstream activator of multiple antiapoptotic signaling molecules and networks, including the signal transducer and activator of transcription 5 (STATS) protein, phosphatidylinositol (PI) 3-kinase/AKT/mammalian target of rapamycin (mTOR) pathway, and nuclear factor kappa B (NF-κB). Further, BTK associates with the death receptor Fas via its kinase and pleckstrin homology (PH) domains and prevents the interaction of Fas with Fas-associated protein with death domain (FADD), which is essential for the recruitment and activation of caspase-8/FLICE by Fas during the apoptotic signal. This impairment by BTK prevents the assembly of a proapoptotic death-inducing signaling complex (DISC) after Fas ligation.

BTK is abundantly expressed in malignant cells from patients with B-cell precursor (BCP)-acute lymphoblastic leukemia (ALL, the most common form of cancer in children and adolescents), chronic lymphocytic leukemia (CLL), and non-Hodgkin's lymphoma (NHL). Consequently, BTK has emerged as an important molecular target for treatment of B-lineage leukemias and lymphomas.

There are a number of BTK inhibitors in the clinic. The first molecule that has been approved is ibrutinib (1-(3-(4-amino-3-(4-phenoxphenyl)-1H-pyrazolo[3,4-d]pyrimidin-1-yl)piperidin-1-yl)prop-2-en-1-one disclosed in WO2008/039218). Ibrutinib is currently used to treat B cell cancers like mantle cell lymphoma, chronic lymphocytic leukemia, and Waldenstrom's macroglobulinemia. Ibrutinib is used as a first line treatment in those with chronic lymphocytic leukemia (CLL) who require treatment and are newly diagnosed and may also be used in CLL that relapses. Ibrutinib is further used to treat Waldenstrom's macroglobulinemia, and as a second-line treatment for mantle cell lymphoma (MCL), marginal zone lymphoma, and chronic graft vs host disease. Recently, Acalabrutinib has been approved by the FDA for treatment of mantle cell lymphoma (www.fda.gov/news-events/press-announcements/fda-approves-new-treatment-adults-mantle-cell-lymphoma).

Both primary (inherent) and secondary (acquired) resistance to ibrutinib has been reported in various lymphomas, including CLL and MCL (Kaur, 2017, Ann Hematol. doi:10.1007/s00277-017-2973-2). A variety of patent publication have therefore suggested to use ibrutinib as part of a combination therapy with other treatment modalities (see e.g. US2017239351, WO 2017/023815 A1, US 2018/0153892 A1, US 2017360796, US 2015105409, US 2017354655, US 2017224819 and US 2016/0303130).

B-cell lymphoma 2 (Bcl-2) is an anti-apoptotic protein localized to the outer membrane of the mitochondria, where it plays an important role in promoting cellular survival and inhibiting the actions of pro-apoptotic proteins. This protein belongs to the Bcl-2 family, a family composed by two groups of highly conserved proteins (anti-apoptotic and pro-apoptotic proteins). The homeostatic balance between these two groups of proteins is necessary for the cells to regulate cell death and cell survival (Scheffold et al., Recent Results Cancer Res. 2018; 212:215-242. doi: 10.1007/978-3-319-91439-8_11; Moia et al., Expert Rev Hematol. 2018 May; 11(5):391-402. doi: 10.1080/17474086.2018.1456332.)

The upregulation of Bcl-2 protein has been well-documented in multiple haematological malignancies. For example, in CLL patients, the increased levels of Bcl-2 protein can be a consequence of an epigenetic dysregulation of the Bcl-2 gene promoter, or—mainly—as a result of deletions at locus 13q14. This deleted region includes two Bcl-2 repressors, microRNAs 15a and 16-1, which binds to the mRNA of the gene and inhibit the translation of the Bcl-2 protein (Scheffold et al., 2018, supra; Moia et al., 2018, supra).

In the last years, new therapies targeting the anti-apoptotic Bcl-2 proteins have been developed. Venetoclax (ABT-199) was approved by the FDA as a second-line treatment for CLL with 17p deletion (Deeks, 2016, Drugs. doi: 10.1007/s40265-016-0596-x). Despite the promising results obtained in CLL patients treated with venetoclax (Moia et al., 2018, supra), some studies report the eventually development of venetoclax resistances. Zhao et al. (Cancer Cell. 2019; 35(5):752-766.e9. doi: 10.1016/j.ccell.2019.04.005) discovered that during venetoclax treatment, some populations of cells lose the region of the chromosome 18 that contains the Bcl-2 gene, contributing to the survival of these cell population. Tahir et al. (BMC Cancer. 2017; 17(1):399. doi: 10.1186/s12885-017-3383-5.) and Chiron et al. (Oncotarget. 2015; 6(11):8750-9) independently identified in several cell lines another mechanism of venetoclax resistance based on an upregulation of the anti-apoptotic MCL-1 and BCL-XL proteins, and a downregulation of the anti-apoptotic BAX, BIM and NOXA proteins. In MCL, cases, venetoclax resistance was additionally achieved through an increment in the apoptotic threshold. Finally, Chiron et al. (2015; supra) also reported that the CD40-CD40L interaction between the pathological cells and the stroma may also circumvent venetoclax activity by means of a potent activation of both classical and alternative NF-kB pathways, which mediate BCL-XL upregulation.

Human CC motif receptor 7 (hereinafter referred to as “CCR7”) is a seven transmembrane-spanning domain G-protein coupled receptor (GPCR) that was originally found to be expressed in a lymphocyte-selective manner by EBV infection (Birkenbach et al., 1993, J. Virol. 67: 2209-2220). CCR7 selectively binds two chemokines named CCL19 and CCL21. In homeostasis and inflammation, CCR7 is expressed on naïve T and B lymphocytes, central memory T cells (TCM), some subsets of natural killer cells (NK cells), semimature and mature DCs, and plasmacytoid DCs (Forster R, et al. Cell 1999; 99: 23-33.; Comerford I, et al. Cytokine Growth Factor Rev. 2013 June; 24(3):269-83). In these leukocyte subsets CCR7 controls migration, organization, and activation.

Alfonso-Pérez et al. (J Leukoc Biol. 2006 June; 79(6):1157-65) disclose that an anti-human CCR7 antibody mediates a potent, complement-dependent cytotoxicity (CDC) against CLL cells while sparing normal T lymphocytes from the same patients and that the antibody blocked the in vitro migration of CLL cells in response physiological ligands of CCR7. WO 2007/003426 therefore discloses the use of anti-human CCR7 antibodies for treating tumors expressing a CCR7 receptor, including hematological tumors such as CLL and MCL.

Patrussi et al. (Cancer Res. 2015 Oct. 1; 75(19):4153-63) disclose that ibrutinib treatment of CLL cells resulted in a significant downregulation in surface CCR7, on the basis of which combining an anti-CCR7 antibody with ibrutinib would not be expected to improve its therapeutic efficacy.

In addition, De Rooij et al. (Blood, 2012; 119(11):2590-4) reported that ibrutinib treatment of primary CLL cells impaired integrin-mediated adhesion and/or migration mediated by CCR7 activation. It is therefore conceivable that ibrutinib would reduce homing of CLL and B-cell lymphoma cells to the secondary lymphoid organs (SLOs) where its ligands are produced. That said, combining antibodies targeting CCR7 with ibrutinib would not be expected to improve the therapeutic inhibition of migration of malignant cells to the SLOs. Moreover, due to the loss of CCR7 on target cell surface, target cell killing triggered by antibodies is unlikely.

In addition to BTK-inhibitors, B-cell malignancies (and CLL in particular) can be treated with inhibitors of the B-cell lymphoma 2 (Bcl-2) protein, an oncogenic protein known to inhibit apoptosis (Gentile et al., Expert Opin Investig Drugs. 2017; 26(11):1307-1316). It has been long accepted that, in cancer, CCR7 plays a role in T-cell protection from apoptosis (Kim et al., Clin Cancer Res. 2005; 11(21):7901-10). Moreover, in this study the evaluation through multicolor flow cytometry significantly correlated higher levels of Bcl-2 with higher expression of CCR7 in these cells and, activation of CCR7 led to phosphorylation of P3K/Akt and the subsequent increment in Bcl-2 expression. It is therefore imaginable that by treating patients with Bcl-2 inhibitors would reduce CCR7-induced anti-apoptotic effects thus precluding the combination of these compounds with anti-CCR7 antibodies.

### SUMMARY OF THE INVENTION

It is therefore an object of the present invention to provide for medicaments and therapeutic approaches that overcome the disadvantages of the prior art approaches for preventing and treating hematological malignancies, in particular B cell malignancies such as CLL. In particular it is an object of the present invention to improve survival rate in such B cell malignancies.

In a first aspect, the invention relates to an anti-CCR7 antibody for use in the treatment of a hyperproliferative hematological disorder, wherein in the disorder is at least one of: a) a disorder that is treated with at least one of a Bruton's tyrosine kinase (BTK) inhibitor and a B-cell lymphoma 2 (Bcl-2) inhibitor; b) a disorder that has relapsed after treatment with at least one of a BTK inhibitor and a Bcl-2 inhibitor; and, c) a disorder that is refractory to treatment with at least one of a BTK inhibitor and a Bcl-2 inhibitor. In the treatment the anti-CCR7 antibody can be administered simultaneously, separately or sequentially with at least one of a BTK inhibitor and a Bcl-2 inhibitor. The hyperproliferative hematological disorder to be treated in accordance with the invention preferably is a disorder wherein the hyperproliferating cells are cells of the B cell lineage, wherein more preferably, the disorder is a B-cell hematological malignancy, most preferably lymphoma or leukemia. Preferably, the hematological malignancy to be treated in accordance with the invention is a hematological malignancy selected from the group consisting of acute lymphocytic leukemia (ALL), acute myeloid leukemia (AML), acute monocytic leukemia (AMoL), chronic lymphocytic leukemia (CLL), high-risk CLL, small lymphocytic lymphoma (SLL), high-risk SLL, multiple myeloma (MM), non-Hodgkin's lymphoma (NHL), mantle cell lymphoma (MCL), follicular lymphoma (FL), Waldenstrom's macroglobulinemia (VVM), diffuse large B-cell lymphoma (DLBCL), marginal zone lymphoma (MZL), Burkitt's lymphoma (BL), hairy cell leukemia (HCL), Richter's transformation and T-cell prolymphocytic leukemia (T-PLL).

The anti-CCR7 antibody for use in a treatment in accordance with the invention preferably has an IC₅₀ of no more than 100 nM for inhibiting at least one of CCR7-dependent intracellular signaling and CCR7 receptor internalization, by at least one CCR7-ligand selected from CCL19 and CCL21. The anti-CCR7 antibody, further preferably inhibits CCR7-dependent intracellular signaling without substantial agonistic effects. A preferred anti-CCR7 antibody for use in a treatment in accordance with the invention has a K_{d} for the N-terminal extracellular domain of human CCR7 that is not more than a factor 20 higher than the K_{d} of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2. The anti-CCR7 antibody preferably is a chimeric, humanized or human antibody. A preferred chimeric, humanized or human anti-CCR7 antibody for use in a treatment in accordance with the invention is an antibody having the HVRs of the anti-human CCR7 antibody of which the amino acid sequence of the heavy chain variable domain is SEQ ID NO: 1 and of which the amino acid sequence of the light chain variable domain is SEQ ID NO: 2.

A preferred BTK inhibitor for use in a treatment in accordance with the invention is ibrutinib, zanabrutinib or acalabrutinib, and a preferred Bcl-2 inhibitor for use in a treatment in accordance with the invention is venetoclax or navitoclax.

The hyperproliferative hematological disorder to be treated in accordance with the invention preferably is a disorder in a treatment-naïve patient, more preferably, the hyperproliferative hematological disorder is a disorder in a patient who is naïve to the treatment with at least one of a BTK inhibitor, a Bcl-2 inhibitor and an anti-CCR7 antibody.

In one embodiment, the hyperproliferative hematological disorder to be treated in accordance with the invention is a hyperproliferative hematological disorder that is refractory to and/or has relapsed after treatment with a chemotherapeutic agent other than a BTK inhibitor, a Bcl-2 inhibitor and an anti-CCR7 antibody, whereby the chemotherapeutic agent preferably is one or more of fludarabine, cyclophosphamide, idelalisib, an anti-CD20 antibody, wherein preferably the anti-CD20 antibody is rituximab, obinituzumab, ocrelizumab, veltuzumab or ofatumumab, or an anti-CD52 antibody, wherein preferably the anti-CD52 antibody is alemtuzumab.

In another embodiment, the hyperproliferative hematological disorder to be treated in accordance with the invention is a hyperproliferative hematological disorder that is refractory to and/or has relapsed after treatment with at least one of a BTK inhibitor and a Bcl-2 inhibitor.

### DESCRIPTION OF INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the methods and materials described.

For purposes of the present invention, the following terms are defined below.

As used herein, the singular forms “a,” “an” and “the” include plural referents unless the context clearly dictates otherwise. For example, a method for administrating a drug or an agent includes the administration of a plurality of molecules (e.g. 10's, 100's, 1000's, 10's of thousands, 100's of thousands, millions, or more molecules) as well as a plurality of drugs or agents.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

As used herein “cancer” and “cancerous”, refer to or describe the physiological condition in mammals that is typically characterized by unregulated cell growth. Cancer is also referred to as malignant neoplasm.

As used herein, “in combination with” is intended to refer to all forms of administration that provide a first drug together with a further (second, third) drug. The drugs may be administered simultaneous, separate or sequential and in any order, unless specified otherwise. Drugs administered in combination have concerted biological activity in the subject to which the drugs are delivered.

As used herein “simultaneous” administration refers to administration of more than one drug at the same time, but not necessarily via the same route of administration or in the form of one combined formulation. For example, one drug may be provided orally whereas the other drug may be provided intravenously during a patient's visit to a hospital. Separate includes the administration of the drugs in separate form and/or at separate moments in time, but again, not necessarily via the same route of administration. Sequentially indicates that the administration of a first drug is followed, immediately or in time, by the administration of the second drug.

As used herein, “to comprise” and its conjugations is used in its non-limiting sense to mean that items following the word are included, but items not specifically mentioned are not excluded. It also encompasses the more limiting “to consist of”.

As used herein “compositions”, “products” or “combinations” useful in the methods of the present disclosure include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure or invention normally comprise the drugs (alone or in combination) and one or more suitable pharmaceutically acceptable excipients.

As used herein, “an effective amount” is meant the amount of an agent required to ameliorate the symptoms of a disease relative to an untreated patient. The effective amount of active agent(s) used to practice the present invention for therapeutic treatment of a cancer varies depending upon the manner of administration, the age, body weight, and general health of the subject. Ultimately, the attending physician or veterinarian will decide the appropriate amount and dosage regimen. Such amount is referred to as an “effective” amount. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The term “antibody” is used in the broadest sense and specifically covers, e.g. single anti-CCR7 monoclonal antibodies, including antagonist, neutralizing antibodies, full length or intact monoclonal antibodies, anti-CCR7 antibody compositions with polyepitopic specificity, polyclonal antibodies, multivalent antibodies, single chain anti-CCR7 antibodies and fragments of anti-CCR7 antibodies (see below), including Fab, Fab′, F(ab′)2 and Fv fragments, diabodies, single domain antibodies (sdAbs), as long as they exhibit the desired biological and/or immunological activity. The term “immunoglobulin” (Ig) is used interchangeable with antibody herein. An antibody can be human and/or humanized.

The term “anti-CCR7 antibody” or “an antibody that binds to CCR7” refers to an antibody that is capable of binding CCR7 with sufficient affinity such that the antibody is useful as a diagnostic and/or therapeutic agent in targeting CCR7. Preferably, the extent of binding of an anti-CCR7 antibody to an unrelated, non-CCR7 protein is less than about 10% of the binding of the antibody to CCR7 as measured, e.g., by a radioimmunoassay (RIA) or ELISA. In certain embodiments, an antibody that binds to CCR7 has a dissociation constant (K) of 1 mM, ≤100 nM, ≤10 nM, ≤1 nM, or ≤0.1 nM. In certain embodiments, anti-CCR7 antibody binds to an epitope of CCR7 that is conserved among CCR7 from different species. It is further understood herein that the term “anti-CCR7 antibody” as used herein includes fragments of the antibody that bind to CCR7.

An “isolated antibody” is one which has been identified and separated and/or recovered from a component of its natural environment.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the a and y chains and four CH domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-33 amino acids separated by shorter regions of extreme variability called “hypervariable regions” (HVRs) that are 7-25 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), complement dependent cytotoxicity (CDC) and antibody dependent cellular phagocytosis (ADCP).

An “intact” antibody is one which comprises an antigen-binding site as well as a CL and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

A “naked antibody” for the purposes herein is an antibody that is not conjugated to a cytotoxic moiety or radiolabel.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments. In one embodiment, an antibody fragment comprises an antigen binding site of the intact antibody and thus retains the ability to bind antigen.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, in contrast to polyclonal antibody preparations which include different antibodies directed against different determinants (epitopes). Monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old-World Monkey, Ape etc.), and human constant region sequences.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, a few framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise typically two variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma and Immunol., 1:105-115 (1998); Harris, Biochem. Soc. Transactions, 23:1035-1038 (1995); Hurle and Gross, Curr. Op. Biotech., 5:428-433 (1994).

The term “hypervariable region”, “HVR”, when used herein refers to the regions of an antibody variable domain which are hypervariable in sequence and/or form structurally defined loops that are responsible for antigen binding. Generally, antibodies comprise six hypervariable regions; three in the VH (H1, H2, H3), and three in the VL (L1, L2, L3). A number of hypervariable region delineations are in use and are encompassed herein. The hypervariable regions generally comprise amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with Honneger, A. and Plunkthun, A. J. (Mol. Biol. 309:657-670 (2001)). The hypervariable regions/CDRs of the antibodies of the invention are preferably defined and numbered in accordance with the IMGT numbering system.

“Framework” or “FR” residues are those variable domain residues other than the hypervariable region residues herein defined.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

A “K_{d}” or “K_{d} value” can be measured by using surface plasmon resonance assays using a BIAcore™-2000 or a BIAcore™-3000 (BIAcore, Inc., Piscataway, N.J.) at 25° C. with immobilized antigen CM5 chips at ˜10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10mM sodium acetate, pH 4.8, into 5 μg/ml (˜0.2 NM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25p1/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco spectrophotometer (ThermoSpectronic) with a stir red cuvette.

An “on-rate” or “rate of association” or “association rate” or “kₒₙ” according to this invention can also be determined with the same surface plasmon resonance technique described above using a BIAcore™-2000 or a BIAcore™-3000 (BIAcore, Inc., Piscataway, N.J.) as described above.

An antibody “which binds” an antigen of interest, e.g. a polypeptide CCR7 antigen target, is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labelled target. In this case, specific binding is indicated if the binding of the labelled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target (which may be determined as described above) of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cell-mediated phagocytosis (ADCP); down regulation of cell surface receptors (e.g. B cell receptor); and B cell activation.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC;

phagocytosis; down regulation of cell surface receptors (e.g. B cell receptor; BCR), etc. Such effector functions generally require the Fc region to be combined with a binding domain (e.g. an antibody variable domain) and can be assessed using various assays as disclosed, for example, in definitions herein.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. (USA) 95:652-656 (1998). WO 2000/42072 (Presta) describes antibody variants with improved or diminished binding to FcRs. See also, e.g., Shields et al. J. Biol. Chem. 9(2):6591-6604 (2001).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al. (1996, J. Immunol. Methods 202:163), may be performed. Antibody variants with altered Fc region amino acid sequences (antibodies with a variant Fc region) and increased or decreased C1q binding capability are described, e.g. in U.S. Pat. No. 6,194,551 B1 and WO 1999/51642. See also, e.g., Idusogie et al. (2000, J. Immunol. 164: 4178-4184). One such substitution that increases C1q binding, and thereby an increases CDC activity, is the E333A substitution, which can advantageously be applied in the antibodies of the invention.

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The terms “sequence identity” or “sequence similarity” means that two (poly)peptide or two nucleotide sequences, when optimally aligned, preferably over the entire length (of at least the shortest sequence in the comparison) and maximizing the number of matches and minimizes the number of gaps such as by the programs ClustalW (1.83), GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides, the default scoring matrix used is nwsgapdna and for proteins the default scoring matrix is Blosum62 (Henikoff & Henikoff, 1992, PNAS 89, 915-919). A preferred multiple alignment program for aligning protein sequences of the invention is ClustalW (1.83) using a blosum matrix and default settings (Gap opening penalty:10; Gap extension penalty: 0.05). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have substantially different overall lengths, local alignments, such as those using the Smith Waterman algorithm, are preferred. Alternatively, percentage similarity or identity may be determined by searching against public databases, using algorithms such as FASTA, BLAST, etc.

### DETAILED DESCRIPTION OF INVENTION

The invention is based on the unexpected finding that contrary to previous reports that BTK inhibitors, such as ibrutinib, can down-regulate or induce complete loss of the CCR7 receptor in chronic lymphocytic leukemia (CLL) cells (thus precluding the use of anti-CCR7 antibodies in this patients), the inventors, studying a large cohort of CLL samples, have found no (or only slight) differences in CCR7 expression levels between CLL patients treated and not treated with ibrutinib. Furthermore, CCR7 expression levels in refractory/relapsed patients where similar or even higher compared to control untreated patients. In addition, the inventors have unexpectedly found that BTK inhibitors showed only minor reduction of CCR7 migration in CLL cells whereas anti-CCR7 antibodies completely blocked this process. Finally, they found that the use of anti-CCR7 antibodies effectively killed CLL cells derived from patients on treatment with BTK inhibitors and from patients with BTK-inhibitors relapsed/refractory disease.

In the same cohort of CLL patients, inventors have observed that CCR7 expression is preserved in CLL patients after treatment with Bcl-2 inhibitors, such as venetoclax, and have confirmed that anti-CCR7 in vitro therapy in these patients led to inhibition of CCR7 functionality and induced target cell killing.

This opens up the possibility to use monoclonal antibodies (mAbs) against CCR7, i.e. antibodies which recognize an epitope in a CCR7 receptor and which preferably are capable of inhibiting CCR7-dependent intracellular signaling and are capable of in vivo killing and/or blocking migration, activation, proliferation and/or dissemination of CCR7 tumor cells, for treating CLL and other hyperproliferative hematological disorders in combination with at least one of a BTK- and a Bcl-2-inhibitor, as well as for treating such disorders that have relapses after treatment with at least one of a BTK- and a Bcl-2-inhibitor or that have become refractory to such treatment.

In a first aspect therefore, the invention relates to anti-CCR7 antibody, for use in the treatment of a hyperproliferative hematological disorder. The hyperproliferative hematological disorder, preferably is at least one of: a) a disorder that is treated with a Bruton's tyrosine kinase (BTK) inhibitor; b) a disorder that is treated with a B-cell lymphoma 2 (Bcl-2) inhibitor; c) a disorder that is treated with a combination of a BTK inhibitor and a Bcl-2 inhibitor; d) a disorder that has relapsed after treatment with a BTK inhibitor; e) a disorder that has relapsed after treatment with a BTK inhibitor; f) a disorder that has relapsed after treatment with a combination of a BTK inhibitor and a Bcl-2 inhibitor; g) a disorder that is refractory to treatment with a BTK inhibitor; h) a disorder that is refractory to treatment with a Bcl-2 inhibitor; and, i) a disorder that is refractory to treatment with a combination of a BTK inhibitor and a Bcl-2 inhibitor.

Thus, in one embodiment, the hyperproliferative hematological disorder is treated with a combination of a BTK inhibitor and an anti-CCR7 antibody. In another embodiment, the hyperproliferative hematological disorder is treated with a combination of a Bcl-2 inhibitor and an anti-CCR7 antibody. In another embodiment, the hyperproliferative hematological disorder is treated with a combination of a BTK inhibitor, a Bcl-2 inhibitor, and an anti-CCR7 antibody. In the treatment, the anti-CCR7 antibody can be administered simultaneously, separately or sequentially with the BTK inhibitor and/or the Bcl-2 inhibitor.

The hyperproliferative hematological disorder to be treated in accordance with the invention, is a disorder in a “subject” or “patient” whereby these terms refer to all animals classified as mammals and includes, but is not restricted to, primates and humans. The subject to be treated preferably is a male or female human and can be of any age or race. The treatment of the subject or patient includes treatment in the first line or second line, or third line.

The term “combination” as used herein is understood to refer to a combination therapy (as opposed to a monotherapy) wherein treatment comprises the use or administration of an anti-CCR7 antibody and the use or administration of at least one of a BTK inhibitor and a Bcl-2-inhibitor. Thus, in a combination therapy according to the invention the components of the combination can be administered simultaneously, separately or sequentially. The components of the combination can thus be formulated in a single composition, or the components can be formulated in at least two separate formulations. The combination can be a single product, comprising a single composition or comprising the components formulated in at least two separate formulations. Alternatively, the combination can be at least two different products that can be from one or more than one supplier.

In some embodiment, the hyperproliferative hematological disorder to be treated in accordance with the invention, preferably with a combination of an anti-CCR7 antibody and at least one of a BTK-inhibitor and a Bcl-2-inhibitor, is a disorder in a treatment-naïve patient, e.g. a patient having received no prior treatment or at least no prior chemotherapeutic treatment, whereby chemotherapeutic treatment has a broad meaning as defined below. More preferably, the treatment-naïve patient is a patient not having received prior treatment with a Bcl-2 inhibitor in case the anti-CCR7 antibody is administered in combination with a BTK inhibitor, or a patient not having received prior treatment with a BTK-inhibitor in case the anti-CCR7 antibody is administered in combination with a Bcl-2 inhibitor. Thus, “not having received prior treatment” is herein to be understood as that the individual had not been treated with the respective inhibitor, prior to the start of the respective combination therapy. Combination treatment of treatment-naïve patients with an anti-CCR7 antibody and at least one of a BTK-inhibitor and a Bcl-2-inhibitor reduces the risk that the patient becomes refractory to one of the components in the treatment and relapses.

In some embodiment, the hyperproliferative hematological disorder to be treated in accordance with the invention, preferably with a combination of an anti-CCR7 antibody and at least one of a BTK-inhibitor and a Bcl-2-inhibitor, is a disorder in a patient who is at least naïve to treatment with at least one of a BTK inhibitor, a Bcl-2 inhibitor and an anti-CCR7 antibody. The patient who is at least naïve to treatment with at least one of a BTK inhibitor, a Bcl-2 inhibitor and an anti-CCR7 antibody, can however be a patient who is refractory to and/or has relapse after treatment with a chemotherapeutic agent other than at least one of a BTK inhibitor, a Bcl-2 inhibitor and an anti-CCR7 antibody.

Thus, in some embodiment, the patient is refractory to and/or has relapsed after treatment with a chemotherapeutic agent other than at least one of a BTK inhibitor, a Bcl-2 inhibitor and an anti-CCR7 antibody. It is understood herein that the term “other chemotherapeutic agent” has a broad meaning which includes biological therapeutic agents such as antibodies and other proteins, antisense molecules, gene therapy vectors and cellular therapies.

In some embodiments, the “other chemotherapeutic agent” is selected from the group consisting of mitotic inhibitors, alkylating agents, antimetabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidilate synthase inhibitors, non-BTK tyrosine kinase inhibitors, P13K-delta tyrosine kinase inhibitors, EGF inhibitors, VEGF inhibitors, CDK inhibitors, SRC inhibitors, c-Kit inhibitors, Her1/2 inhibitors, inhibitors of myc, anti-tumor antibodies, monoclonal antibodies directed against growth factor receptors, protein kinase modulators, radioactive isotopes, immunotherapies, glucocorticoids, and combinations thereof.

In some embodiments, the “other chemotherapeutic agent” is an anticancer agent selected from the group consisting of DNA interactive agents, such as cisplatin or doxorubicin; topoisomerase II inhibitors, such as etoposide; topoisomerase I inhibitors, such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors, EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); other protein kinase modulators and combinations thereof. Other anti-cancer agents that could be used in the methods of the invention will be known to those skilled in the art of oncology.

In some embodiments, the “other chemotherapeutic agents” is selected from the group consisting of a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, (−)-7-methylomuralide, (+/−)-7-methylomuralide, PS-341, vinyl sulfone tripeptide inhibitors, ritoavir, PI-083, lenalidomide, and combinations thereof.

In some embodiments, the “other chemotherapeutic agent” is a combination of chemotherapies such as, e.g., “CHOP” (a combination including (i) cyclophosphamide, such as cytoxan, (ii) doxorubicin or other topoisomerase II inhibitors such as adriamycin, (iii) vincristine or other vincas such as oncovin; and (iv) a steroid such as hydrocortisone or prednisolone); “R-CHOP” (a combination including rituxan, cyclophosphamide, doxorubicin, vincristine, and prednisone); “ICE” (a combination including ifosfamide, carboplatin, and etoposide); “R-ICE” (a combination including rituxan, ifosfamide, carboplatin, and etoposide); “R-ACVBP” (a combination of rituximab, doxorubicin, cyclophosphamide, vindesine, bleomycin and prednisone); “DA-EPOCH-R” (a combination of dose-adjusted etoposide, doxorubicin, cyclophosphamide, vincristine, prednisone and rituximab); “R-bendamustine” (a combination of bendamustine and rituximab); “GemOx or R-GemOx” (a combination of semcitabine and oxaliplatin, with or without rituximab); and “DHAP” (a combination including dexamethasone, cytarabine, and cisplatin).

In a preferred embodiment, “other chemotherapeutic agent” is fludarabine, cyclophosphamide, idelalisib, an anti-CD20 antibody, wherein preferably the anti-CD20 antibody is rituximab, obinituzumab, ocrelizumab, veltuzumab or ofatumumab, or an anti-CD52 antibody, wherein preferably the anti-CD52 antibody is alemtuzumab, or combinations thereof.

In another embodiment of the invention, the hyperproliferative hematological disorder is refractory to and/or has relapsed after treatment with a BTK inhibitor, whereby preferably the BTK inhibitor is as herein defined below. As exemplified herein a hyperproliferative hematological disorder can be refractory to and/or relapsed after treatment with a BTK inhibitors such as ibrutinib, acalabrutinib and zanabrutinib. The hyperproliferative hematological disorder to be treated in accordance with the invention can be a disorder that is refractory to and/or has relapsed after treatment with a BTK inhibitor when the inhibitor was used as sole agent in the treatment of the disorder (i.e. used as a monotherapy). Alternatively, the hyperproliferative hematological disorder to be treated in accordance with the invention can be a disorder that is refractory to and/or has relapsed after treatment with a BTK inhibitor when the inhibitor was used in combination with another chemotherapeutic agent (i.e. used as a combination therapy). The other chemotherapeutic agent can be an “other chemotherapeutic agent” as herein defined above. In a preferred embodiment, “other chemotherapeutic agent” is one or more of fludarabine, cyclophosphamide, idelalisib, an anti-CD20 antibody, wherein preferably the anti-CD20 antibody is rituximab, obinituzumab, ocrelizumab, veltuzumab or ofatumumab, or an anti-CD52 antibody, wherein preferably the anti-CD52 antibody is alemtuzumab. In another embodiment, the hyperproliferative hematological disorder to be treated in accordance with the invention can be a disorder that is refractory to and/or has relapsed after successive treatments, each treatment with a different chemotherapeutic agent or a combination thereof and wherein one of the treatments comprised the use of a BTK inhibitor (in mono- or in combination therapy). In this embodiment, the different chemotherapeutic agent or a combination thereof can be an “other chemotherapeutic agent” as herein defined above.

In yet another embodiment of the invention, the hyperproliferative hematological disorder is refractory to and/or has relapsed after treatment with a Bcl-2 inhibitor, whereby preferably the Bcl-2 inhibitor is as herein defined below. As exemplified herein, a hyperproliferative hematological disorder can be refractory to and/or relapsed after treatment with a Bcl-2 inhibitor such as venetoclax. The hyperproliferative hematological disorder to be treated in accordance with the invention can be a disorder that is refractory to and/or has relapsed after treatment with a Bcl-2 inhibitor when the inhibitor was used as sole agent in the treatment of the disorder (i.e. used as a monotherapy). Alternatively, the hyperproliferative hematological disorder to be treated in accordance with the invention can be a disorder that is refractory to and/or has relapsed after treatment with a Bcl-2 inhibitor when the inhibitor was used in combination with another chemotherapeutic agent (i.e. used as a combination therapy). The other chemotherapeutic agent can be an “other chemotherapeutic agent” as herein defined above. In a preferred embodiment, “other chemotherapeutic agent” is one or more of fludarabine, cyclophosphamide, ibrutinib, idelalisib, an anti-CD20 antibody, wherein preferably the anti-CD20 antibody is rituximab, obinituzumab, ocrelizumab, veltuzumab or ofatumumab, or an anti-CD52 antibody, wherein preferably the anti-CD52 antibody is alemtuzumab. In another embodiment, the hyperproliferative hematological disorder to be treated in accordance with the invention can be a disorder that is refractory to and/or has relapsed after successive treatments, each treatment with a different chemotherapeutic agent or a combination thereof and wherein one of the treatments comprised the use of a Bcl-2 inhibitor (in mono- or in combination therapy). In this embodiment, the different chemotherapeutic agent or a combination thereof can be an “other chemotherapeutic agent” as herein defined above.

In again another embodiment of the invention, the hyperproliferative hematological disorder is refractory to and/or has relapsed after treatment with the combination of a BTK inhibitor and Bcl-2 inhibitor, whereby preferably the BTK inhibitor and the Bcl-2 inhibitor is as herein defined below. As exemplified herein a hyperproliferative hematological disorder can be refractory to and/or relapsed after treatment with the combination of a BTK inhibitors such as ibrutinib, acalabrutinib and zanabrutinib and a Bcl-2 inhibitor such as venetoclax. The hyperproliferative hematological disorder to be treated in accordance with the invention can be a disorder that is refractory to and/or has relapsed after treatment with the combination of a BTK inhibitor and a Bcl-2 inhibitor when the combination of these inhibitors was used as sole combination in the treatment of the disorder (i.e. no further agents were used in the therapy). Alternatively, the hyperproliferative hematological disorder to be treated in accordance with the invention can be a disorder that is refractory to and/or has relapsed after treatment with the combination of a BTK inhibitor and a Bcl-2 inhibitor when the inhibitor was used in combination with another chemotherapeutic agent (i.e. used as a combination therapy). The other chemotherapeutic agent can be an “other chemotherapeutic agent” as herein defined above. In a preferred embodiment, “other chemotherapeutic agent” is one or more of fludarabine, cyclophosphamide, idelalisib, an anti-CD20 antibody, wherein preferably the anti-CD20 antibody is rituximab, obinituzumab, ocrelizumab, veltuzumab or ofatumumab, or an anti-CD52 antibody, wherein preferably the anti-CD52 antibody is alemtuzumab. In another embodiment, the hyperproliferative hematological disorder to be treated in accordance with the invention can be a disorder that is refractory to and/or has relapsed after successive treatments, each treatment with a different chemotherapeutic agent or a combination thereof and wherein one of the treatments comprised the use of a combination of a BTK inhibitor and a Bcl-2 inhibitor (as such or in combination with another chemotherapeutic agent). In this embodiment, the different chemotherapeutic agent or a combination thereof can be an “other chemotherapeutic agent” as herein defined above.

The hyperproliferative hematological disorder to be treated in accordance with the invention is a disorder wherein the hyperproliferating cells preferably express the CCR7 receptor. In particular when the anti-CCR7 antibody is administered simultaneously, separately or sequentially with at least a BTK inhibitor, the hyperproliferating cells preferably express a Bruton's tyrosine kinase. In particular when the anti-CCR7 antibody is administered simultaneously, separately or sequentially with at least a Bcl-2 inhibitor the hyperproliferating cells preferably express a B-cell lymphoma 2 protein. In some embodiments, the hyperproliferating cells express or overexpress at least one of CD20 and CD52. In another embodiment, the hyperproliferating cells have lost or reduced expression of at least CD20, as in some patients CD20 expression on the hyperproliferating cells is down regulated after BTK treatment whereas CCR7 is still expressed.

In one embodiment, the hyperproliferative hematological disorder to be treated in accordance with the invention is a disorder wherein the hyperproliferating cells are cells of the B cell lineage. In some embodiments, the disorder associated with excessive B-cell proliferation is cancer. In some embodiments, the disorder is cancer. In some embodiments, the cancer is a B-cell hematological malignancy. In certain embodiments, the B-cell hematological malignancy is lymphoma or leukemia.

In some embodiments, the hematological malignancy is selected from the group consisting of acute lymphocytic leukemia (ALL), acute myeloid leukemia (AML), acute monocytic leukemia (AMoL), chronic lymphocytic leukemia (CLL), high-risk CLL, small lymphocytic lymphoma (SLL), high-risk SLL, multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), Burkitt's lymphoma (BL), hairy cell leukemia (HCL), Richter's transformation and T-cell prolymphocytic leukemia (T-PLL).

In some embodiments, the hematological malignancy is selected from the group consisting of chronic lymphocytic leukemia (CLL), small lymphocytic lymphoma (SLL), non-Hodgkin's lymphoma (NHL), mantle cell lymphoma (MCL), follicular lymphoma (FL), diffuse large B-cell lymphoma (DLBCL), marginal zone lymphoma (MZL) and T-cell prolymphocytic leukemia (T-PLL).

In some embodiments, the hematological malignancy is selected from the group consisting of Burkitt's lymphoma, non-Burkitt's high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, plasma cell myeloma, plasmacytoma, mediastinal (thymic) large B cell lymphoma, intravascular large B cell lymphoma, primary effusion lymphoma, Hodgkin's lymphoma and lymphomatoid granulomatosis.

### The Anti-CCR7 Antibody

The anti-CCR7 antibody or antigen-binding fragment thereof, for use in the present invention can be any antigen binding protein that specifically binds to CCR7. An antigen binding protein of the invention that binds to CCR7 preferably is an anti-CCR7 antibody in the broadest sense as defined herein above, including e.g. anti-CCR7 antibodies, antibody fragments, antibody derivatives, antibody muteins, and antibody variants. An anti-CCR7 antibody of the invention preferably is an isolated antibody. Preferably, an anti-CCR7 antibody of the invention binds to a primate CCR7, more preferably to human CCR7. Reference amino acid sequences of human CCR7 are e.g. NP_001288643, NP_001288645, NP_001288646, NP_001288647, NP_001829, NP_001288642 and NP_031745. Amino acids 1 to 24 of this sequence comprise the membrane translocation signal peptide, which is cleaved off during expression. Amino acids 25 to 59 of human CCR7 make up the N-terminal extracellular domain, which domain comprises sulfated tyrosine residues in position Y32 and Y41. Various allelic variants are known for human CCR7 with one or more amino acid substitutions compared to the above-mentioned reference sequences. “Human CCR7” in the present invention includes these allelic variants, at least in as far as the variants have an extracellular domain and the function of CCR7. An anti-CCR7 antibody for use in the invention preferably specifically binds to the N-terminal extracellular domain of a CCR7, preferably a human CCR7.

An anti-CCR7 antibody for use in the invention preferably is a neutralizing antibody that inhibits CCR7-dependent intracellular signaling, CCR7-dependent functions, and/or CCR7 receptor internalization by at least one CCR7 ligand selected from CCL19 and CCL21. An anti-CCR7 antibody preferably has an IC₅₀ that is not higher than 150, 100, 80, 50, 30, 25, 20, 15, 10, 5 or 3 nM for inhibiting CCR7-dependent intracellular signaling and/or CCR7 receptor internalization by at least one CCR7 ligand selected from CCL19 and CCL21, as can e.g. be determined in assay as described in the Examples herein. Alternatively, the maximal IC₅₀ of the antibody is defined by reference to the IC₅₀ of a reference anti-CCR7 antibody when tested in the same assay. Thus, preferably an anti-CCR7 antibody of the invention has an IC₅₀ that is not more than a factor 10, 5, 2, 1.5, 1.2, 1.1 or 1.05 higher than the IC₅₀ of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

An anti-CCR7 antibody of the invention preferably inhibits CCR7-dependent intracellular signaling CCR7 as described above, without substantial agonistic effects, more preferably without detectable agonistic effects, as can e.g. be determined in assay as described in the Examples herein.

An anti-CCR7 antibody for use in the invention preferably has a minimal affinity for the N-terminal extracellular domain of a CCR7, preferably a human CCR7. The minimal affinity of the antibody is herein preferably defined by reference to the K_{d} of a reference anti-CCR7 antibody when tested in the same assay. Thus, preferably an anti-CCR7 antibody of the invention has a K_{d} for the N-terminal extracellular domain of human CCR7 that is not more than a factor 100, 50, 20, 10, 5, 2, 1.5, 1.2, 1.1 or 1.05 higher than the K_{d} of a reference anti-CCR7 antibody for the N-terminal extracellular domain of human CCR7, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2. It is understood herein that an antibody with a K_{d} that is not more than a factor 10 higher times than the K_{d} of a reference is an antibody that has an affinity that is not less than a factor 10 lower than the affinity of the reference antibody. Thus, if the reference antibody has a K_{d} of 1×10⁻⁹ M, the antibody in question has a K_{d} of 1×10⁻⁸ M or less.

Examples of anti-CCR7 antibodies with one or more of the above-defined characteristics and suitable for use in the present invention include e.g. the monoclonal antibodies described in U.S. Pat. No. 8,865,170, WO 2009/139853, US 20150344580 (WO2013184200), US 2016031997, US 2017342155 (WO 2014/151834) and US 2018237529 (WO 2017/025569), all of which are incorporated herein by reference.

A preferred anti-CCR7 antibody for use in the present invention is an antibody that specifically binds to an epitope comprising or consisting of the amino acid sequence “ZxLFE”, wherein Z is a sulfated tyrosine and x can be any amino acid and F can be replaced by a hydrophobic amino acid. The antibody of the invention thus preferably specifically binds to an epitope comprising or consisting of the amino acids sequence “ZTLFE” in positions 41 to 45 in the N-terminal extracellular domain of human CCR7. The antibody preferably is specific for human CCR7. Such a preferred anti-CCR7 antibody preferably has at least a minimal affinity for human CCR7 or for a synthetic antigen comprising the “ZTLFE” epitope, preferably for the synthetic antigen SYM1899 as described in the Examples herein. Preferably therefore, the anti-CCR7 antibody has a K_{d} of 1×10⁻⁸ M, 5×10⁻⁹ M, 2×10⁻⁹ M, 1.8×10⁻⁹ M, 1×10⁻⁹ M, 1×10⁻¹⁰ M or 1×10⁻¹¹ M or less preferably for the synthetic antigen SYM1899. Alternatively, the minimal affinity of the antibody is defined by reference to the K_{d} of a reference anti-CCR7 antibody when tested in the same assay. Thus, preferably an anti-CCR7 antibody of the invention has a K_{d} for human CCR7 or for a synthetic antigen comprising the “ZTLFE” epitope (preferably the synthetic antigen SYM1899 as described in the Examples herein) that is not more than a factor 10, 5, 2, 1.5, 1.2, 1.1 or 1.05 higher than the K_{d} of a reference anti-CCR7 antibody for the antigen, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2. It is understood herein that an antibody with a K_{d} that is not more than a factor 10 higher times than the K_{d} of a reference is an antibody that has an affinity that is not less than a factor 10 lower than the affinity of the reference antibody. Thus, if the reference antibody has a K_{d} of 1×10⁻⁹ M, the antibody in question has a K_{d} of 1×10⁻⁸ M or less.

An anti-CCR7 antibody for use in the invention preferably binds to human CCR7 or to a synthetic antigen comprising the “ZTLFE” epitope (preferably the synthetic antigen SYM1899 as described in the Examples herein; SEQ ID NO: 3) with k_{off} rate constant that has a maximal upper limit. Preferably therefore, the anti-CCR7 antibody of the invention has a k_{off} rate constant that is 1×10⁻³, 1×10⁻⁴ or 1×10⁻⁵ s⁻¹ or less. Alternatively, the maximal k_{off} rate constant of the antibody is defined by reference to the k_{off} rate constant of a reference anti-CCR7 antibody when tested in the same assay. Thus, preferably an anti-CCR7 antibody of the invention binds to human CCR7 or to a synthetic antigen comprising the “ZTLFE” epitope (preferably the synthetic antigen SYM1899 as described in the Examples herein) that is not more than a factor 10, 5, 2, 1.5, 1.2, 1.1 or 1.05 higher than the k_{off} rate constant of a reference anti-CCR7 antibody for the antigen, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

One such preferred antibody for use in the present invention is an antibody having the HVRs of the reference mouse anti-human CCR7 antibody of which the amino acid sequence of the heavy chain variable domain is SEQ ID NO: 1 and of which the amino acid sequence of the light chain variable domain is SEQ ID NO: 2, which HVRs are defined in US 2018237529, incorporated by reference herein.

An anti-CCR7 antibody for use in the invention can be a chimeric antibody, e.g. mouse-human antibody. However, preferably the antibody is a humanized or human antibody.

A humanized antibody for use in the invention preferably elicits little to no immunogenic response against the antibody in a subject to which the antibody is administered. For example, a humanized antibody for use in the invention elicits and/or is expected to elicit a human anti-mouse antibody response (HAMA) at a substantially reduced level compared to the original mouse an antibody, e.g. comprising the sequence of SEQ ID NO: 1 and 2 in a host subject. Preferably, the humanized antibody elicits and/or is expected to elicit a minimal or no human anti-mouse antibody response (HAMA). Most preferably, an antibody of the invention elicits anti-mouse antibody response that is at or less than a clinically acceptable level.

Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. In practice, humanized antibodies are typically human antibodies in which some hypervariable region residues and possibly some framework region (FR) residues are substituted by residues from analogous sites in rodent antibodies. The choice of human variable domains, both light and heavy, to be used in making the humanized antibodies is very important to reduce immunogenicity retaining the specificity and affinity for the antigen. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Suns et al., J. Immunol., 151:2296 (1993); Chothia et al., J. Mol. Biol, 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies are humanized, with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. A humanized anti-CCR7 antibody, according to any of the above embodiments of the invention, preferably comprises a heavy chain constant region that is IgG1, IgG2, IgG3 or IgG4 region. A humanized anti-CCR7 antibody according to any of the above embodiments of the invention, preferably comprises a functional Fc region possessing at least one effector function selected from the group consisting of: C1q binding, complement dependent cytotoxicity; Fc region binding, antibody-dependent cell-mediated cytotoxicity and phagocytosis.

A preferred humanized antibody for use in the present invention is an antibody of which the amino acid sequence of the heavy chain variable domain is SEQ ID NO: 4 and of which the amino acid sequence of the light chain variable domain is SEQ ID NO: 5, as e.g. described in US 2018237529. A more preferred humanized antibody for use in the present invention is an antibody of which the amino acid sequence of the heavy chain variable domain is SEQ ID NO: 4, of which the amino acid sequence of the light chain variable domain is SEQ ID NO: 5, and of which the amino acid sequence of the heavy chain constant region is SEQ ID NO: 10, as e.g. described in US 2018237529, and referred to as “CAP-100” herein.

As an alternative to humanization, human antibodies can be generated. By “human antibody” is meant an antibody containing entirely human light and heavy chains as well as constant regions, produced by any of the known standard methods. For example, transgenic animals (e.g., mice) are available that are capable, upon immunization, of producing a full repertoire of human antibodies in the absence of endogenous immunoglobulin production. For example, it has been described that the homozygous deletion of the antibody heavy-chain joining region PH gene in chimeric and germ-line mutant mice results in the complete inhibition of endogenous antibody production. Transfer of the human germ-line immunoglobulin gene array in such germ line mutant mice will result in the production of human antibodies after immunization. See, e.g., Jakobovits et al., Proc. Nat. Acad. Sci. USA, 90:255 1 (1993); Jakobovits et al., Nature, 362:255-258 (1993). Alternatively, phage display technology (McCafferty et al., Nature 348:552-553 (1990)) can be used to produce human antibodies and antibody fragments in vitro, from immunoglobulin variable (V) domain gene repertoires from donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-57 1 (1993). Human antibodies may also be generated by in vitro activated B cells or SCID mice with its immune system reconstituted with human cells. Once a human antibody is obtained, its coding DNA sequences can be isolated, cloned and introduced into an appropriate expression system i.e. a cell line, preferably from a mammal, which subsequently express and liberate it into a culture media from which the antibody can be isolated.

A preferred human antibody for use in the present invention is an antibody of which the amino acid sequence of the heavy chain variable domain is SEQ ID NO: 6 and of which the amino acid sequence of the light chain variable domain is SEQ ID NO: 7 or 8, as e.g. described in US 2016031997 (WO 2014/151834), or any one of the human anti-CCR7 antibodies described in US 20150344580 (WO2013/184200), such as the R707 antibody of which the amino acid sequence of the heavy chain is SEQ ID NO: 11 and of which the amino acid sequence of the light chain is SEQ ID NO: 12. Other preferred human antibodies for use in the present invention are human antibodies that comprise all of the VH regions of the anti CCR7 antibodies that are described in US2013195869 (WO2012043533) and WO2018142322.

Functional fragments of antibodies which bind to a CCR7 receptor that are included for use within the present invention retain at least one binding function and/or modulation function of the full-length antibody from which they are derived. Preferred functional fragments retain an antigen-binding function of a corresponding full-length antibody (e.g., the ability to bind a mammalian CCR7 receptor). Particularly preferred functional fragments retain the ability to inhibit one or more functions characteristic of a mammalian CCR7 receptor, such as a binding activity and/or blocking a signaling activity, and/or stimulation of a cellular response. For example, in one embodiment, a functional fragment can inhibit the interaction of CCR7 with one or more of its ligands and/or can inhibit one or more receptor-mediated functions.

In some embodiments, an anti-CCR7 antibody of the invention comprises a light chain and/or a heavy chain antibody constant region. Any antibody constant regions known in the art can be used. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. An anti-CCR7 antibody of the invention can thus have constant regions of any isotype, i.e. including IgG, IgM, IgA, IgD, and IgE constant regions as well as IgG1, IgG2, IgG3, or IgG4 constant regions. In one embodiment, the light or heavy chain constant region is a fragment, derivative, variant, or mutein of a naturally occurring constant region. Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al. (2002, Methods Mol. Bio1.178:303-16). Accordingly, the anti-CCR7 antibodies of the invention include those comprising, for example, one or more of the variable domain sequences disclosed herein and having a desired isotype (e.g., IgA, IgGI, IgG2, IgG3, IgG4, IgM, IgE, and IgD), as well as Fab or F(ab′)2 fragments thereof. Moreover, if an IgG4 is desired, it may also be desired to introduce a point mutation (CPSCP->CPPCP) in the hinge region as described in Bloom et al. (1997, Protein Science 6:407) to alleviate a tendency to form intra-H chain disulfide bonds that can lead to heterogeneity in the IgG4 antibodies.

An anti-CCR7 antibody of the invention preferably comprises a functional Fc region possessing at least one effector function selected from the group consisting of: C1q binding, complement dependent cytotoxicity; Fc receptor binding, antibody-dependent cell-mediated cytotoxicity and phagocytosis.

An anti-CCR7 antibody of the invention can be modified to improve effector function, e.g. so as to enhance ADCC and/or CDC of the antibody. This can be achieved by introducing one or more amino acid substitutions in an Fc region of an antibody. A preferred substitution in the Fc region of an antibody of the invention is a substitution that increases C1q binding, and thereby increases CDC activity, such as e.g. described in Idusogie et al. (2000, J. Immunol. 164: 4178-4184). A preferred substitution in the Fc region that increases C1q binding is the E333A substitution.

Glycosyl groups added to the amino acid backbone of glycoproteins e.g. antibodies are formed by several monosaccharides or monosaccharide derivatives in resulting in a composition which can be different in the same antibody produced in cell from different mammals or tissues. In addition, it has been shown that different composition of glycosyl groups can affect the potency in mediating antigen-dependent cell-mediated cytotoxicity (ADCC) and/or complement dependent cytotoxicity (CDC) of the antibody. Therefore, it is possible to improve those properties by mean of studying the pattern of glycosylation of antibodies from different sources. An example of such approach is Niwa et al. (2004, Cancer Res, 64(6):2127-33).

Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al. (1992, J. Exp Med. 176:1191-1195) and Shopes, (1992, Immunol. 148:2918-2922). Homodimeric antibodies with enhanced anti-tumor activity may also be prepared using heterobifunctional cross-linkers as described in Wolff et al. (1993, Cancer Research 53:2560-2565). Alternatively, an antibody which has dual Fc regions can be engineered and may thereby have enhanced complement lysis and ADCC capabilities. See Stevenson et al. (1989, Anti-Cancer Drug Design 3:2 19-230). In order to increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

A preferred anti-CCR7 antibody of the invention comprises a heavy chain constant region of the human allotype G1m17,1 (see Jefferis and Lefranc (2009) MAbs Vol. 1 Issue 4, pp 1-7), which heavy chain constant region comprises the amino acid sequence of SEQ ID NO: 9. More preferably, the heavy chain constant region of the human allotype G1m17,1 in the antibody of the invention comprises an E333A substitution, which heavy chain constant region comprises the amino acid sequence of SEQ ID NO: 10.

Anti-CCR7 antibodies for use in the invention can be prepared by any of a number of conventional techniques. They will usually be produced in recombinant expression systems, using any technique known in the art. See e.g. Shukla and Thommes (2010, “Recent advances in large-scale production of monoclonal antibodies and related proteins”, Trends in Biotechnol. 28(5):253-261), Harlow and Lane (1988) “Antibodies: A Laboratory Manual”, Cold Spring Harbor Laboratory Press, Cold Spring Harbor, N.Y., and Sambrook and Russell (2001) “Molecular Cloning: A Laboratory Manual (3^{rd} edition), Cold Spring Harbor Laboratory, Cold Spring Harbor Laboratory Press, NY. Any expression system known in the art can be used to make the recombinant polypeptides of the invention. In general, host cells are transformed with a recombinant expression vector that comprises DNA encoding a desired polypeptide.

### The BTK Inhibitor

The invention relates to new combinations involving inhibitors of Bruton's tyrosine kinase (BTK), referred to as BTK inhibitors. Such inhibitors are broadly known in the art and are commercially available for the treatment of B cell cancers like mantle cell lymphoma, chronic lymphocytic leukemia, and Waldenstrom's macroglobulinemia. An example of a commercially available BTK inhibitor is ibrutinib, also known as Imbruvica (trade name), which is 1-[(3R)-3-[4-Amino-3-(4-phenoxyphenyl)-1H-pyrazolo[3,4-d]pyrimidin-1-yl]piperidin-1-yl]prop-2-en-1-one.

The activity of BTK inhibitors can be measured using the Immobilized Metal Assay for Phosphochemicals (IMAP). IMAP is a homogeneous fluorescence polarization (FP) assay based on affinity capture of phosphorylated peptide substrates. IMAP uses fluorescein-labeled peptide substrates that, upon phosphorylation by a protein kinase, bind to so-called IMAP nanoparticles, which are derivatized with trivalent metal complexes. Binding causes a change in the rate of the molecular motion of the peptide, and results in an increase in the FP value observed for the fluorescein label attached to the substrate peptide.

BTK inhibition can also be determined in B cell lines such as Ramos cells or in primary cell assays, e.g., PBMC or whole blood from mammals such as human, monkey, rat, or mouse; or isolated splenocytes from monkey, rat, or mouse. Inhibition of BTK activity can be investigated measuring anti-IgM-induced MIP1 β production (Ramos, PBMC, splenocytes), H₂O₂-induced BTK and PLCv2 phosphorylation (Ramos cells), or anti-IgM-induced B cell proliferation, or CD86 expression on primary B cells (PBMC and splenocytes). Regulation of BTK activity can also be determined on human, monkey, rat or mouse mast cells following activation FCER induced degranulation, cytokine production and CD63 induced cell surface expression. Furthermore, regulation of BTK activity can be determined on CD14+ monocytes differentiated following treatment with M-CSF to osteoclasts and activated with RANKL. Activity of BTK inhibitors can be investigated in mouse splenocytes following administration in vivo. In a typical experiment, mice can be sacrificed 3 h following compound administration. Spleens can be extracted from the treated mice for splenocyte isolation. Splenocytes can be plated in 96 well culture plates and stimulated with anti-IgM, without further addition of compounds. Anti-IgM-induced B cell stimulation and inhibition thereof by BTK inhibitors can be measured by B cell proliferation, MIP1 β production or CD86 expression on CD19+ splenocyte B cells. Further assays are known to a skilled person, such as use of mouse collagen induced arthritis model as described in WO2013010869, or use of the rat OVX model as described in WO2013010869.

Preferred BTK inhibitors are selective for BTK or are substantially selective for BTK. More preferably, BTK inhibitors are selective over Src-family kinases, or are selective for BTK and kinases having a cysteine residue in an amino acid sequence position of the tyrosine kinase that is homologous to the amino acid sequence position of cysteine 481 in BTK. BTK inhibitors can form a covalent bond with this cysteine or can otherwise interact with it.

BTK inhibitors generally comprise a ring system featuring two or more nitrogen atoms, in addition to further affinity elements. In preferred embodiments, the BTK inhibitor is of general formula (1)

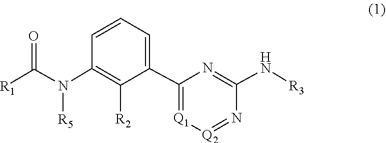

wherein

Q₁ and Q₂ are each independently N or C—R4, wherein preferably one of Q₁ and Q₂ is N or CH, and the other is C—R₄,

R₁ represents a substituted or unsubstituted aryl group, a substituted or unsubstituted heterocyclic ring, a substituted or unsubstituted heterocyclic fused ring, or a substituted or unsubstituted alkynyl group,

R₂ represents a hydrogen atom, a halogen atom, a substituted or unsubstituted lower alkyl group, or a substituted or unsubstituted alkoxy group,

R₃ represents a substituted or unsubstituted aryl group, a substituted or unsubstituted heterocyclic ring, or a substituted or unsubstituted heterocyclic fused ring,

R₄ represents a hydrogen atom, a substituted or unsubstituted lower alkyl group, a substituted or unsubstituted alkoxy group, a substituted or unsubstituted amino group, or a halogen atom, and

R₅ represents a hydrogen atom, a substituted or unsubstituted lower alkyl group, or R₁ and R₅ may be combined to form a saturated or unsaturated 5- to 6-membered ring, thereby forming a multiply fused ring,

or the BTK inhibitor is of general formula (2)

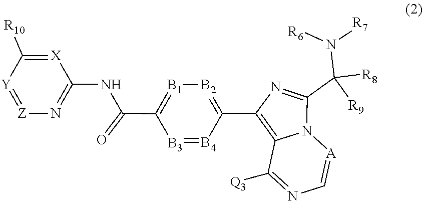

wherein

Q₃ is an amino group or a lower alkyl group, preferably an amino group or methyl,

X is CH, N, O or S;

Y is C(R₁₁), N, O or S;

Z is CH, N or a bond;

A is CH or N;

B₁ is N or C(R₁₂);

B₂ is N or C(R₁₃);

B₃ is N or C(R₁₄);

B₄ is N or C(R₁₅);

R₆ is C(═O)R₁₆, S(═O)R₁₇, S(═O)₂R₁₈ or (C₁₋₆)alkyl optionally substituted with R₁₉;

R₇ is H, (C₁₋₃)alkyl or (C₃₋₇)cycloalkyl;

R₈ is H, (C₁₋₆)alkyl or (C₃₋₇)cycloalkyl); or R₇ and R₈ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;

R₉ is H or (C₁₋₃)alkyl;

R₁₀ is H, halogen, cyano, (C₁₋₄)alkyl, (C₁₋₃)alkoxy, (C₃₋₆)cycloalkyl, any alkyl group of which is optionally substituted with one or more halogen; or R₁₀ is (C₆₋₁₀)aryl or (C₂₋₆) heterocycloalkyl;

R₁₁ is H or (C₁₋₃)alkyl; or Rio and Ru together may form a (C₃₋₇)cycloalkenyl or (C₂₋₆)heterocycloalkenyl, each optionally substituted with (C₁₋₃)alkyl or one or more halogens;

R₁₂ is H, halogen, CF₃, (C₁₋₃)alkyl or (C₁₋₃)alkoxy;

R₁₃ is H, halogen, CF₃, (C₁₋₃)alkyl or (C₁₋₃)alkoxy; or R₁₂ and R₁₃ together with the carbon atoms they are attached to, form (C₆₋₁₀)aryl or (C₁₋₉) heteroaryl;

R₁₄ is H, halogen, (C₁₋₃)alkyl or (C₁₋₃)alkoxy;

R₁₅ is H, halogen, (C₁₋₃)alkyl or (C₁₋₃)alkoxy;

R₁₆ is independently selected from the group consisting of (C₁₋₆)alkyl, (C₂₋₆)alkenyl and (C₂₋₆)alkynyl, where each alkyl, alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or R₁₆ (C₁₋₃)alkyl-C(O)—S—(C₁₋₃)alkyl; or R₁₆ is (C₁₋₅)heteroaryl optionally substituted with one or more substituents selected from the group consisting of halogen and cyano;

R₁₇ and R₁₈ are independently selected from the group consisting of (C₂₋₆)alkenyl and (C₂₋₆) alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or a (C₁₋₅)heteroaryl optionally substituted with one or more substituents selected from the group consisting of halogen and cyano; and

R₁₉ is independently selected from the group consisting of halogen, cyano, (C₂₋₆)alkenyl and (C₂₋₆)alkynyl, both optionally substituted with one or more substituents selected from the group consisting of hydroxyl, (C₁₋₄)alkyl, (C₃₋₇)cycloalkyl, (C₁₋₄)alkylamino, di[(C₁₋₄)alkyl]amino, (C₁₋₃)alkoxy, (C₃₋₇)cycloalkoxy, (C₆₋₁₀)aryl, (C₁₋₅)heteroaryl and (C₃₋₇)heterocycloalkyl; with the proviso that:

0 to 2 atoms of X, Y, Z can simultaneously be a heteroatom; when one atom selected from X, Y is O or S, then Z is a bond and the other atom selected from X, Y cannot be O or S; when Z is C or N then Y is C(R₁₁) or N and X is C or N; 0 to 2 atoms of B₁, B₂, B₃, and B₄ are N;

or the BTK inhibitor is of general formula (3)

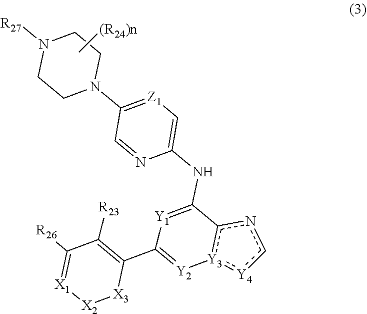

wherein

the solid/dash line indicates a single or double bond;

X₁ is CR₂₀ or N;

X₂ is CR₂₁ or N;

X₃ is CR₂₂ or N; where none, one, or two of X₁, X₂, and X₃ are N;

Y₁ and Y₂ are independently selected from CH and N;

Y₃ is C or N;

Y₄ is CR₂₅, N or NH; where one or two of Y₁, Y₂, Y₃, and Y₄ are N;

R₂₀, R₂₁, and R₂₂ are independently selected from H, F, Cl, —NH₂, —NHCH₃, —N(CH₃)₂, —OH, —OCH₃, —OCH₂CH₃, —OCH₂CH₂OH, and C₁₋₃ alkyl optionally substituted with F, Cl, CN, —NH₂, —NHCH₃, —N(CH₃)₂, —OH, —OCH₃, —OCH₂CH₃, and —OCH₂CH₂OH;

R₂₃ is selected from H, F, Cl, CN, —CH₂OH, —CH(CH₃)OH, —C(CH₃)₂OH, —CH(CF₃)OH, —CH₂F, —CHF₂, —CH₂CHF₂, —CF₃, —C(O)NH₂, —C(O)NHCH₃, —C(O)N(CH₃)₂, —NH₂, —NHCH₃, —N(CH₃)₂, —NHC(O)CH₃, —OH, —OCH₃, —OCH₂CH₃, —OCH₂CH₂OH, cyclopropyl, cyclopropylmethyl, 1-hydroxycyclopropyl, imidazolyl, pyrazolyl, 3-hydroxy-oxetan-3-yl, oxetan-3-yl, and azetidin-1-yl;

R₂₄ is selected from C₁₋₂alkyl, —CH₂OH, —CH₂F, —CHF₂, —CF₃, —CN, and —CH₂CH₂OH; or two R₂₄ groups form a 3-, 4-, 5-, or 6-membered carbocyclic or heterocyclic ring;

or an R₂₄ group and an R₂₇ group form a 3-, 4-, 5-, or 6-membered carbocyclic or heterocyclic ring;

n is 0, 1, 2, 3, or 4;

R₂₅ is selected from H, Cl, C₁₋₂alkyl, —CH₂CH₂OH, —CH₂F, —CHF₂, —CF₃, —NH₂, —NHCH₃, —N(CH₃)₂, —OH, C₁₋₂alkoxy, and —OCH₂CH₂OH;

R₂₆ is a moiety of general formula (3₂₆)

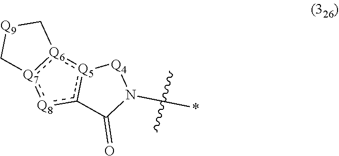

wherein

the solid/dash line indicates a single or double bond;

Q₄ is —CH₂—, —CH₂CH₂—, —CH═CH—, —CH═N—, or —NH—,

Q₅, Q₆, and Q₇ are each independently C or N;

Q₈ is C, S, N, or C(halogen);

Q₉ is —CH₂—, —CH₂CH₂—, —C(CH₃)₂—, —C(halogen)₂CH₂—, or C₄₋₆cycloalkyl such as cyclopentyl attached at two non-adjacent carbon atoms, preferably the 1- and 3-position;

R₂₇ is selected from H, —CH₃, —S(O)₂CH₃, cyclopropyl, azetidin-3-yl, oxetan-3-yl, and morpholin-4-yl;

Z is CR₂₈ or N; and

R₂₈ is selected from H, F, Cl, C₁₋₂alkyl, —CH₂CH₂OH, —NH₂, —NHCH₃, —N(CH₃)₂, —OH, C₁₋₂alkoxy, and —OCH₂CH₂OH;

or the BTK inhibitor is of general formula (4)

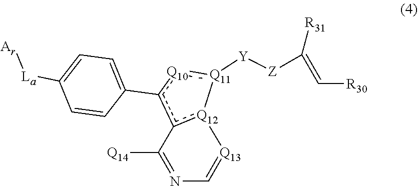

wherein

the solid/dash line indicates a single or double bond;

Q₁₀, Q₁₁, Q₁₂, and Q₁₃ are each independently N or NH depending on valency or C or CH depending on valency, wherein at least one of Q₁₀, Q₁₁, Q₁₂, and Q₁₃ is N or NH and at least one is C or CH;

Q₁₄ is NH₂ or CH₃;

Lₐ is CH₂, O, NH or S;

Aᵣ is a substituted or unsubstituted aryl, or a substituted or unsubstituted heteroaryl;

Y is an optionally substituted group selected from the group consisting of alkyl, heteroalkyl, cycloalkyl, heterocycloalkyl, aryl and heteroaryl;

Z is C(═O), OC(═O), NR₂₉C(═O), C(═S), S(═O)ₓ, OS(═O)ₓ or NR₂₉S(═O)ₓ, where x is 1 or 2;

R₂₉ is H or (C₁₋₆)alkyl; and

R₃₀ and R₃₁ are each independently H; or R₃₀ and R₃₁ taken together form a bond;

or the BTK inhibitor is of general formula (5)

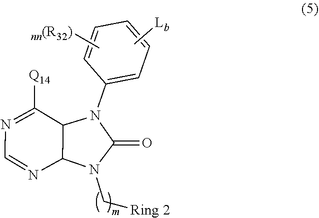

wherein

Q₁₅ is NH₂ or CH₃;

L_{b} represents O, S, SO, SO₂, NH, C(O), CH₂O, OCH₂, CH₂, or CH(OH);

R₃₂ represents halogen, C₁₋₄ alkyl, C₁₋₄ alkoxy, C₁₋₄ haloalkyl, or C₁₋₄ haloalkoxy;

Ring 1 represents a 4- to 7-membered cyclic group, which may be substituted by from one to five substituents each independently selected from the group consisting of halogen, C₁₋₄ alkyl, C₁₋₄ alkoxy, nitrile, C₁₋₄ haloalkyl, and C₁₋₄ haloalkoxy, wherein when two or more substituents are present on Ring 1, these substituents may form a 4- to 7-membered cyclic group together with the atoms in Ring 1 to which these substituents are bound;

Ring 2 represents a 4- to 7-membered saturated heterocycle, which may be substituted by from one to three KR₃₃;

K represents a bond, C₁₋₄ alkylene, C(O), C(O)CH₂, CH₂C(O), C(O)O, or SO₂;

R₃₃ represents C₁₋₄ alkyl, C₂₋₄ alkenyl, or C₂₋₄ alkynyl, each of which may be substituted by from one to five substituents each independently selected from the group consisting of NR₃₄R₃₅, halogen, CONR₃₆R₃₇, CO₂R₃₈, and OR₃₉;

R₃₄ and R₃₅ each independently represent H, or a C₁₋₄ alkyl group which may be substituted by OR₄₀ or CONR₄₁R₄₂; R₃₄ and ₃₅ may, together with the nitrogen atom to which they are bound, form a 4- to 7-membered nitrogenous saturated heterocycle, which may be substituted by an oxo group or OH;

R₃₆ and R₃₇ each independently represent H, C₁₋₄ alkyl, or phenyl;

R₃₈, R₄₀, R₄₁, and R₄₂ each independently represent H or C₁₋₄ alkyl;

R₃₉ represents H, C₁₋₄ alkyl, phenyl, or a benzotriazolyl group;

nn represents an integer from 0 to 4;

m represents an integer from 0 to 2; and

when n is two or more, any R₃₂ may be the same as each other or may differ from one another;

or the BTK inhibitor is of general formula (6)

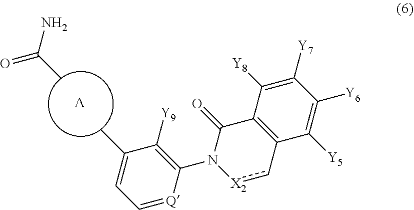

wherein

the solid/dashed line is either a single or double bond;

Ring A is 5-membered heteroaryl or a 5,6-membered bicyclic heteroaryl, wherein the CONH₂ is attached to the 5-membered heteroaryl, each optionally substituted with one or more A′;

A′ is —NHR₄₀ or R₄₄;

R₄₀ is H, —R₄₁, —R₄₁—R₄₂—R₄₃, —R₄₁—R₄₃, or —R₄₂—R₄₃;

R₄₁ is aryl, heteroaryl, cycloalkyl, heterocycloalkyl, or heteroaryl fused with a heterocycloalkyl, each of which is optionally substituted with one or more R¹′ or R¹″;

each R¹′ is independently halo, nitro, cyano, lower alkyl sulfonamido, S(O)₂, or oxo;

each R¹″ is independently lower alkyl, cycloalkyl, heterocycloalkyl, lower alkoxy, amino, or amido, each optionally substituted with one or more R¹′″;

each R¹′″ is independently hydroxy, halo, amino, alkyl amino, dialkyl amino, or heterocycloalkyl;

R₄₂ is —C(═O), —C(═O)O, —C(═O)NR²′, —NHC(═O)O, —C(R²′)₂, O, —C(═NH)NR²>, or —S(═O )₂;

each R²′ is independently H or lower alkyl;

R₄₃ is H or R₄₄;

R₄₄ is lower alkyl, lower haloalkyl, lower alkoxy, amino, lower alkyl amino, lower dialkyl amino, aryl, arylalkyl, alkylaryl, heteroaryl, alkyl heteroaryl, heteroaryl alkyl, cycloalkyl, alkyl cycloalkyl, cycloalkyl alkyl, heterocycloalkyl, alkyl heterocycloalkyl, heterocycloalkyl alkyl, bicyclic cycloalkyl, bicyclic heterocycloalkyl, spirocycloalkyl, or spiroheterocycloalkyl, each of which is optionally substituted with one or more lower alkyl, halo, lower alkyl amino, lower dialkyl amino, hydroxy, hydroxy lower alkyl, lower alkoxy, halo, nitro, amino, amido, acyl, cyano, oxo, guanidino, hydroxyl amino, carboxy, carbamoyl, carbamate, halo lower alkoxy, or halo lower alkyl, wherein two lower alkyl groups may together form a ring;

Q′ is CH or N;

X₂ is CH, N, or N(X₃);

X₃ is lower alkyl;

Y₅ is H, halogen or lower alkyl;

Y₆ is Y₆ₐ, Y_{6b}, Y_{6c}, or Y_{6d},

Y₆ₐ is H or halogen;

Y_{6b} is lower alkyl, optionally substituted with one or more substituents selected from the group consisting of lower haloalkyl, halogen, hydroxy, amino, cyano, and lower alkoxy;

Y_{6c} is lower cycloalkyl, optionally substituted with one or more substituents selected from the group consisting of lower alkyl, lower haloalkyl, halogen, hydroxy, amino, cyano, and lower alkoxy;

Y₆ₐ is amino, optionally substituted with one or more lower alkyl, alkoxy lower alkyl, or hydroxy lower alkyl;

Y₇ is H, halogen or lower alkyl;

Y₈ is H, halogen, lower alkyl, lower haloalkyl, lower alkoxy, or lower hydroxy alkyl; and

Y₉ is H, lower alkyl, or lower hydroxyalkyl;

or the BTK inhibitor is of general formula (7)

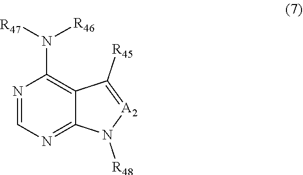

wherein

A₂ is independently selected from N or CR₄₉;

R₄₅ is H, L₂ₐ-(substituted or unsubstituted alkyl), L₂ₐ-(substituted or unsubstituted cycloalkyl), L₂ₐ-(substituted or unsubstituted alkenyl), L₂ₐ-(substituted or unsubstituted cycloalkenyl), L₂ₐ-(substituted or unsubstituted heterocycle), L₂ₐ-(substituted or unsubstituted heteroaryl), or L₂ₐ-(substituted or unsubstituted aryl), where L₂ₐ is a bond, O, S, S(═O), S(═O)₂, C(═O), substituted or unsubstituted C₁₋₆ alkylene, or substituted or unsubstituted C₂₋₆ alkenylene);

R₄₆ and R₄₇ are independently selected from H, lower alkyl and substituted lower alkyl;

R₄₈ is L₃ₐ-Xₐ-L₄ₐ-G, wherein,

L₃ₐ is optional, and when present is a bond, or an optionally substituted group selected from alkylene, heteroalkylene, arylene, heteroarylene, alkylarylene, alkylheteroarylene, or alkylheterocycloalkylene;

Xₐ is optional, and when present is a bond, O, C(═O), S, S(═O), S(═O)₂, NH, NR₅₃, NHC(O), C(O)NH, NR₅₃C(O), C(O)NR₅₃, S(═O)₂NH, NHS(═O)₂, S(═O)₂NR₅₃, NR₅₃S(═O)₂, OC(O)NH, NHC(O)O, OC(O)NR₅₃, NR₅₃C(O)O, C(H)═NO, ON═CH, NR₅₄C(O)NR₅₄, heteroarylene, arylene, NR₅₄C(═NR₅₅)NR₅₄, NR₅₄C(═NR₅₅), C(═NR₅₅)NR₅₄, OC(═NR₅₅), or C(═NR₅₅)O;

L₄ₐ is optional, and when present is a bond, substituted or unsubstituted alkylene, substituted or unsubstituted cycloalkylene, substituted or unsubstituted alkenylene, substituted or unsubstituted alkynylene, substituted or unsubstituted arylene, substituted or unsubstituted heteroarylene, substituted or unsubstituted heterocyclene;

or L₃ₐ, Xₐ and L₄ₐ taken together form a nitrogen containing heterocyclic ring, or an optionally substituted group selected from alkyl, heteroalkyl, aryl, heteroaryl, alkylaryl, alkylheteroaryl, or alkylheterocycloalkyl;

G is selected from the group consisting of

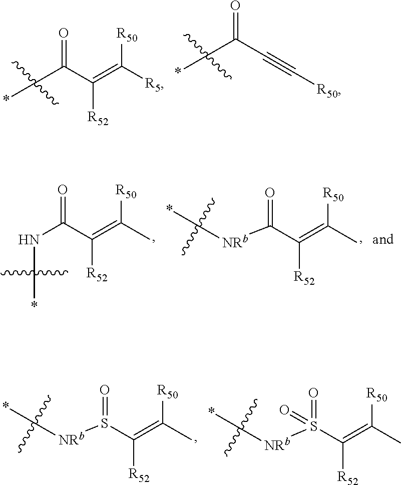

where R^{b} is H, substituted or unsubstituted alkyl, substituted or unsubstituted cycloalkyl; and either of R₅₁ and R₅₂ are H;

R₅₀ is H, substituted or unsubstituted C₁₋₄alkyl, substituted or unsubstituted C₁₋₄heteroalkyl, C₁₋₈alkylaminoalkyl, C₁₋₈hydroxyalkylaminoalkyl, C₁₋₈alkoxyalkylaminoalkyl, substituted or unsubstituted C₃₋₆cycloalkyl, substituted or unsubstituted C₁₋₈alkylC₃₋₆cycloalkyl, substituted or unsubstituted aryl, substituted or unsubstituted C₂₋₈heterocycloalkyl, substituted or unsubstituted heteroaryl, C₁₋₄alkyl(aryl), C₁₋₄alkyl(heteroaryl), C₁₋₈alkylethers, C₁₋₈alkylamides, or C₁₋₄alkyl(C₂₋₈heterocycloalkyl);

or R₅₀ and R₅₂ are H;

R₅₁ is H, substituted or unsubstituted C₁₋₄alkyl, substituted or unsubstituted C₁₋₄heteroalkyl, C₁₋₈alkylaminoalkyl, C₁₋₈hydroxyalkylaminoalkyl, C₁₋₈alkoxyalkylaminoalkyl, substituted or unsubstituted C₃₋₆cycloalkyl, substituted or unsubstituted C₋₈alkylC₃₋₆cycloalkyl, substituted or unsubstituted aryl, substituted or unsubstituted C₂₋₈heterocycloalkyl, substituted or unsubstituted heteroaryl, C₁₋₄alkyl(aryl), C₁₋₄alkyl(heteroaryl), C₁₋₈alkylethers, C₁₋₈alkylamides, or C₁₋₄alkyl(C₂₋₈heterocycloalkyl);

or R₅₁ and R₅₂ taken together form a bond;

R₄₉ is H, halogen, -L₆ₐ-(substituted or unsubstituted C₁₋₃ alkyl), -L₆ₐ-(substituted or unsubstituted C₂₋₄ alkenyl), -L₆ₐ-(substituted or unsubstituted heteroaryl), or -L₆ₐ-(substituted or unsubstituted aryl), wherein Lha is a bond, O, S, S(═O), S(═O)₂, NH, C(O), NHC(O)O, OC(O)NH, NHC(O), or C(O)NH;

R₅₃ is selected from among H, substituted or unsubstituted lower alkyl, and substituted or unsubstituted lower cycloalkyl;

each R₅₄ is independently H, substituted or unsubstituted lower alkyl, or substituted or unsubstituted lower cycloalkyl; or two R₅₄ groups can together form a 5-, 6-, 7-, or 8-membered heterocyclic ring; or

R₅₄ and R₅₅ can together form a 5-, 6-, 7-, or 8-membered heterocyclic ring; or

R₅₅ is selected from H, S(═O)₂R₅₆, S(═O)₂NH₂, C(O)R₅₆, CN, NO₂, heteroaryl, or heteroalkyl;

or the BTK inhibitor is of general formula (8)

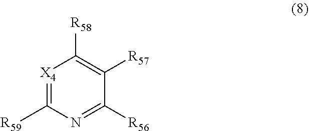

wherein

X₄ denotes CH or N,

R₅₆ denotes NH₂, CONH₂ or H,

R₅₇ denotes Hal, Ar₁₀ or Het_{1b},

R₅₈ denotes NR₆₀[C(R₆₀)₂]ₙHet_{2b}, NR₆₀[C(R₆₀)₂]ₙₙₙCyc, Het_{2b}, O[C(R₆₀)₂]ₙₙₙAr_{2b}, NR_{60[}C(R₆₀)₂]ₙₙAr_{2b}, O[C(R₆₀)₂]ₙₙₙHet_{2b}, NR60(CH₂)ₚNR₆₀R₆₁, O(CH₂)ₚNR₆₀R₆₁ or NR₆₀(CH₂)ₚCR₆₂R₆₃NR₆₀R₆₁,

R₅₉ denotes H, CH₃ or NH₂,

R₆₀ denotes H or alkyl having 1 , 2, 3 or 4 C atoms,

R₆₁ denotes N(R₆₀)₂CH₂CH═CHCONH, Het_{3b}CH₂CH═CHCONH, CH₂═CHCONH(CH₂)ₙₙₙ, Het_{4b}(CH₂)ₙₙₙCOHet_{3b}-diyl-CH₂CH═CHCONH, HC≡CCO, CH₃C≡CCO, CH₂═CH—CO, CH₂═C(CH₃)CONH, CH₃CH═CHCONH(CH₂)ₙₙₙ, N≡CCR₅₂R₅₃CONH(CH₂)ₙₙₙ, Het_{4b}NH(CH₂)ₚCOHet_{3b}-diyl-CH₂CH═CHCONH, Het_{4b}(CH₂)ₚCONH(CH₂CH₂O)ₚ(CH₂)ₚCOHet_{3b}-diyl-CH₂CH═CHCONH, CH₂═CHSO₂, A_{b}CH═CHCO, CH₃CH═CHCO, Het_{4b}(CH₂)ₚCONH(CH₂)ₚHet_{3b}-diyl-CH₂CH═CHCONH, Ar_{3b}CH═CHSO₂, CH₂═CHSO₂NH or N(R₆₀)CH₂CH═CHCO,

R⁶², R⁶³ denote together alkylene having 2, 3, 4, or 5 C atoms,

Ar_{1b} denotes phenyl or naphthyl, each of which is unsubstituted or mono-, di- or trisubstituted by R₆₁, Hal, (CH₂)ₙₙₙNH₂, CONHAr_{3b}(CH₂)ₙₙₙNHCOA_{b}, O(CH₂)ₙₙₙAr_{3b}, OCyC_{b}, A_{b}, COHet_{3b}, OA_{b} and/or OHet_{3b}(CH₂),

Ar_{2b} denotes phenyl, naphthyl or pyridyl each of which is unsubstituted or mono-, di- or trisubstituted by R₆₁, Hal, OAr_{3b}, (CH₂)ₙₙₙNH₂, (CH₂)ₙₙₙNHCOA_{b} and/or Het_{3b},

Ar_{3b} denotes phenyl, which is unsubstituted or mono-, di- or trisubstituted by OH, OA_{b}, Hal, CN and/or A_{b},

Het_{1b} denotes a mono-or bicyclic saturated, unsaturated or aromatic heterocycle having 1 to 4 N, O and/or S atoms, which may be unsubstituted or mono-, di- or trisubstituted by R₆₁, O(CH₂)ₙₙₙAr_{3b} and/or (CH₂)ₙₙₙAr_{3b},

Het_{2b} denotes a mono-or bicyclic saturated heterocycle having 1 to 4 N, O and/or S atoms, which may be unsubstituted or mono-, di- or trisubstituted by R₆₁, Het_{3b}, Cyc_{b}SO₂, OH, Hal, COOH, OA_{b}, COA_{b}, COHet_{3b}, Cyc_{b}CO, SO₂ and/or ═O,

Het_{3b} denotes a monocyclic unsaturated, saturated or aromatic heterocycle having 1 to 4 N, O and/or S atoms, which may be unsubstituted or mono-, di- or trisubstituted by Hal, A and/or ═O,

Het_{4b} denotes a bi- or tricyclic unsaturated, saturated or aromatic heterocycle having 1 to 4 N, O and/or S atoms, which may be unsubstituted or mono-, di-, tri- or tetrasubstituted by A_{b}, NO₂, Hal and/or ═O,

Cyc_{b} denotes cyclic alkyl having 3, 4, 5 or 6 C atoms, which is unsubstituted, monosubstituted or disubstituted by R₆₁ and/or OH and which may comprise a double bond,

A_{b} denotes C₁₋₁₀alkyl, in which 1-7 H atoms may be replaced by F and/or Cl and/or in which one or two non-adjacent CH₂ and/or CH-groups may be replaced by O, NH and/or by N,

Hal denotes F, Cl, Br or I,

nnn denotes 0, 1 , 2, 3 or 4,

denotes 1 , 2, 3, 4, 5 or 6,

or the BTK inhibitor is of general formula (9)

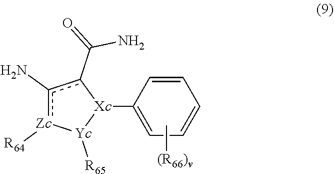

wherein

the solid/dashed line is either a single or double bond;

X_{c}—Y_{c}—Z_{c} is N—C—C and R₆₅ is present, or C—N—N and R₆₅ is absent;

R₆₄ is a 3-8 membered, N-containing ring, wherein the N is unsubstituted or substituted with R₆₇;

R₆₅ is H or lower alkyl, particularly methyl, ethyl, propyl or butyl; or

R₆₄ and R₆₅ together with the atoms to which they are attached, form a 4-8 membered ring, preferably a 5-6 membered ring, selected from cycloalkyl, saturated or unsaturated heterocycle, aryl, and heteroaryl rings unsubstituted or substituted with at least one substituent L_{c}-R₆7;

R₆₆ is in each instance, independently halogen, alkyl, S-alkyl, CN, or OR₆₈;

v is 1, 2, 3, or 4, preferably 1 or 2;

L_{c} is a bond, NH, heteroalkyl, or heterocyclyl;

R₆₇ is COR₆₉, CO₂R₆₉, or SO₂R₆₉, wherein R₆₉ is substituted or unsubstituted alkyl, substituted or unsubstituted alkenyl, substituted or unsubstituted alkynyl;

R₆₈ is H or unsubstituted or substituted heteroalkyl, alkyl, cycloalkyl, saturated or unsaturated heterocyclyl, aryl, or heteroaryl,

or the BTK inhibitor is of general formula (10)

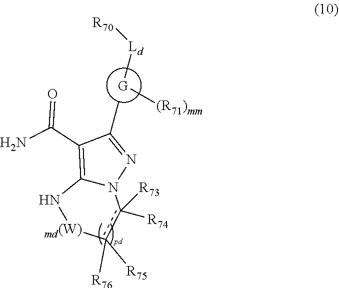

wherein

Ring G is a 5- or 6-membered aromatic ring comprising 0-3 heteroatoms of N, S or O;

each W is independently —(CH₂)— or —C(O)—;

L_{d} is a bond, CH₂, NR₈₁,O, or S;

the solid/dashed line is either a single or double bond, and when a double bond, R₇₄ and R₇₆ are absent;

md is 0, or an integer of 1-4;

mm is 0, or an integer of 1-4, wherein when mm is more than 1, each R₇₁ may be different;

pd is 0, or an integer of 1-2, wherein when pd is 0, md is non-zero, and when pd is more than 1, each R₇₅ and each R₇₆ may be different;

R₇₀, R₇₃, R₇₄, R₇₅, and R₇₆ are each independently H, halogen, heteroalkyl, alkyl, alkenyl, cycloalkyl, aryl, saturated or unsaturated heterocyclyl, heteroaryl, alkynyl, —CN, —NR₈₂R₈₃, OR₈₂, —COR₈₂, —CO₂R₈₂, —CONR₈₂R₈₃, —C(═NR₈₂)NR₈₃R₈₄, —NR₈₂COR₈₃, —NR₈₂CONR₈₃R₈₄, —NR₈₂CO₂R₈₃, —SO₂R₈₂, —NR₈₂SO₂NR₈₃R₈₄, or —NR₈₂SO₂R₈₃, wherein the alkyl, alkenyl, alkynyl, cycloalkyl, heteroaryl, aryl, and saturated or unsaturated heterocyclyl are optionally substituted with at least one substituent R₈₅, wherein (R₈₃ and R₈₄), or (R₈₃ and R₈₅), or (R₈₅ and R₈₆), or (R₈₅ and R₈₅ when pd is 2), together with the atoms to which they are attached, can form a ring selected from cycloalkyl, saturated or unsaturated heterocycle, aryl, and heteroaryl rings optionally substituted with at least one substituent R85;

R₇₁ is halogen, alkyl, —S-alkyl, —CN, —NR₈₂R₈₃, —OR₈₂, —COR₈₂, —CO₂R₈₂, —CONR₈₂R₈₃, —C(═NR₈₂)NR₈₃R₈₄, —NR₈₂COR₈₃, —NR₈₂CONR₈₃R₈₄, —NR₈₂CO2R₈₃, —SO₂R₈₂, —NR₈₂SO₂NR₈₃R₈₄, or —NR₈₂SO₂R₈₃,

R₈₁ is H or lower alkyl;

R₈₂, R₈₃, and R₈₄ are each independently H, heteroalkyl, alkyl, alkenyl, alkynyl, cycloalkyl, saturated or unsaturated heterocyclyl, aryl, or heteroaryl; wherein (R₈₂ and R₈₃), and/or (R₈₃ and R₈₄) together with the atom(s) to which they are attached, each can form a ring selected from cycloalkyl, saturated or unsaturated heterocycle, aryl, and heteroaryl rings optionally substituted with at least one substituent R₈₅;

R₈₅ is halogen, subsituted or unsubstitued alkyl, substituted or unsubstituted alkenyl, subsituted or unsubstitued alkynyl, subsituted or unsubstitued cycloalkyl, subsituted or unsubstitued aryl, subsituted or unsubstitued heteroaryl, subsituted or unsubstitued heterocyclyl, oxo, —CN, —OR₈₆, —NR₈₆R₈₇, —COR₈₆, —CO₂R₈₆, —CONR₈₆R₈₇, —C(═NR₈₆)NR₈₇R₈₈, —NR₈₆COR₈₇, —NR₈₆CONR₈₆R_{87,}—NR₈₆CO₂R₈₇, —SO₂R₈₆, —SO₂aryl, —NR₈₆SO₂NR₈₇R₈₈, or —NR₈₆SO₂R₈₇, wherein R₈₆, R₈₇, and R₈₈ are independently hydrogen, halogen, subsituted or unsubstitued alkyl, substituted or unsubstituted alkenyl, subsituted or unsubstitued alkynyl, subsituted or unsubstitued cycloalkyl, subsituted or unsubstitued aryl, subsituted or unsubstitued heteroaryl, subsituted or unsubstitued heterocyclyl, wherein (R₈₆ and R₈₇), and/or (R₈₇ and R₈₈) together with the atoms to which they are attached, can form a ring selected from cycloalkyl, saturated or unsaturated heterocycle, aryl, and heteroaryl rings

or stereoisomers, tautomers, or pharmaceutically acceptable salts thereof.

In preferred embodiments, the BTK inhibitor is of general formula (1) with variable moieties as defined above, or a stereoisomer, tautomer, or pharmaceutically acceptable salt thereof.

In preferred embodiments, the BTK inhibitor is of general formula (2) with variable moieties as defined above, or a stereoisomer, tautomer, or pharmaceutically acceptable salt thereof.

In preferred embodiments, the BTK inhibitor is of general formula (3) with variable moieties as defined above, or a stereoisomer, tautomer, or pharmaceutically acceptable salt thereof.

In preferred embodiments, the BTK inhibitor is of general formula (4) with variable moieties as defined above, or a stereoisomer, tautomer, or pharmaceutically acceptable salt thereof, more preferably ibrutinib.

In preferred embodiments, the BTK inhibitor is of general formula (5) with variable moieties as defined above, or a stereoisomer, tautomer, or pharmaceutically acceptable salt thereof.

In preferred embodiments, the BTK inhibitor is of general formula (6) with variable moieties as defined above, or a stereoisomer, tautomer, or pharmaceutically acceptable salt thereof.

In preferred embodiments, the BTK inhibitor is of general formula (7) with variable moieties as defined above, or a stereoisomer, tautomer, or pharmaceutically acceptable salt thereof.

In preferred embodiments, the BTK inhibitor is of general formula (8) with variable moieties as defined above, or a stereoisomer, tautomer, or pharmaceutically acceptable salt thereof.

In preferred embodiments, the BTK inhibitor is of general formula (9) with variable moieties as defined above, or a stereoisomer, tautomer, or pharmaceutically acceptable salt thereof.

In preferred embodiments, the BTK inhibitor is of general formula (10) with variable moieties as defined above, or a stereoisomer, tautomer, or pharmaceutically acceptable salt thereof.

A wide variety of pharmaceutically acceptable salts can be formed from BTK inhibitors, including: acid addition salts formed by reacting for example ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like; acid addition salts formed by reacting for example ibrutinib with an inorganic acid, which includes hydrochloric acid, hydrobromic acid, sulfuric acid, nitric acid, phosphoric acid, hydroiodic acid, hydrofluoric acid, phosphorous acid, and the like. An HCl addition salt of a BTK inhibitor is preferred.

Preferred halogens are fluorine, chlorine, and bromine, more preferably fluorine and bromine, most preferably fluorine. An aryl group moiety of the substituted or unsubstituted aryl group is preferably an aryl group having 6 to 14 carbon atoms, and specific examples thereof include phenyl, naphthyl, and indenyl. A heterocyclic ring moiety of the substituted or unsubstituted heterocyclic ring is preferably an alicyclic heterocyclic group or an aromatic heterocyclic group. The alicyclic heterocyclic group includes, for example, 3- to 8-membered heterocyclic group having at least one heteroatom selected from a nitrogen atom, a sulfur atom, and an oxygen atom. Specific examples thereof include pyrrolidinyl, piperidinyl, piperazinyl, morpholinyl, and thiomorpholinyl. The aromatic heterocyclic group includes, for example, 5- or 6-membered monocyclic aromatic heterocyclic group having at least one heteroatom selected from a nitrogen atom, a sulfur atom, and an oxygen atom. Specific examples thereof include imidazolyl, pyrazolyl, thienyl, thiazolyl, and pyridyl. A heterocyclic fused ring moiety of the substituted or unsubstituted heterocyclic fused ring includes, for example, a fused heterocyclic group which is 3- to 8-membered ring-fused bicyclic group and has at least one heteroatom selected from a nitrogen atom, a sulfur atom, and an oxygen atom. Specific examples thereof include tetrahydroisoquinolyl, benzothiophenyl, benzimidazolyl, benzooxazolyl, benzothiazolyl, indolyl, isoquinolyl, and phthalimide. A lower alkyl group moiety of the substituted or unsubstituted lower alkyl group may be any of linear, branched and cyclic alkyl groups having 1 to 3 carbon atoms, and specific examples thereof include a methyl group, an isopropyl group, and a cyclopropyl group. An alkoxy group moiety of the substituted or unsubstituted alkoxy group may be any of linear, branched, or cyclic alkyl group having 1 to 3 carbon atoms, and specific examples thereof include a methoxy group, an ethoxy group, an isopropyloxy group, and a cyclopropyloxy group. The substituted or unsubstituted amino group may be more specifically any of amino groups having linear, branched, or cyclic alkyl group having 1 to 3 carbon atoms, and specific examples thereof include an amino group, a methylamino group, and a dimethylamino group. It is preferably an amino group. An alkynyl group moiety of the substituted or unsubstituted alkynyl group may be any of linear or branched group having 2 to 6 carbon atoms, and specific examples thereof include ethynyl group, propargyl group, 2-butynyl group. A substituted moiety of the substituted or unsubstituted alkynyl group may be any of a substituted or unsubstituted aryl ring, a substituted or unsubstituted heterocyclic ring, or a substituted or unsubstituted heterocyclic fused ring, and specific examples thereof include aryl group.

In preferred embodiments the BTK inhibitor has, as a substituent of the substituted or unsubstituted aryl group, the substituted or unsubstituted heterocyclic ring, the substituted or unsubstituted heterocyclic fused ring, the substituted or unsubstituted lower alkyl group, the substituted or unsubstituted alkoxy group, or the substituted or unsubstituted amino group, one, or two or more of any kind of substituent (s) at any chemically possible position. When the above group has two or more substituents, the respective substituents may be the same or different, and examples of the substituent include a halogen atom, a substituted or unsubstituted alkyl group, a substituted or unsubstituted alkoxy group, a substituted or unsubstituted amino group, a nitro group, a cyano group, a hydroxy group, a substituted or unsubstituted alkylamino group, a substituted or unsubstituted carbamoyl group, a carboxyl group, a formyl group, an acetyl group, a benzoyl group, and a substituted or unsubstituted acylamino group. A multiply fused ring which can be formed by combining R₅ and R₁ to form a saturated or unsaturated 5- to 6-membered ring includes, for example, 3- to 8-membered ring-fused heterocyclic group having heteroatoms such as a nitrogen atom, a sulfur atom, and an oxygen atom. Specific examples thereof include oxoisoquinolyl, oxodihydroisoquinolyl, oxophthalazinyl, and oxothienopyrrolyl.

(C₁₋₂)alkyl means an alkyl group having 1 to 2 carbon atoms, being methyl or ethyl, (C₁₋₃)alkyl means a branched or unbranched alkyl group having 1-3 carbon atoms, being methyl, ethyl, propyl or isopropyl; (C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred; (C₁₋₅)alkyl means a branched or unbranched alkyl group having 1-5 carbon atoms, for example methyl, ethyl, propyl, isopropyl, butyl, isobutyl, sec-butyl, tert-butyl, pentyl and isopentyl, (C₁₋₄)alkyl groups being preferred. (C₁₋₆)Alkyl means a branched or unbranched alkyl group having 1-6 carbon atoms, for example methyl, ethyl, propyl, isopropyl, butyl, tert-butyl, n-pentyl and n-hexyl. (C₁₋₅)alkyl groups are preferred, (C₁₋₄)alkyl being most preferred.

(C₁₋₂)alkoxy means an alkoxy group having 1-2 carbon atoms, the alkyl moiety having the same meaning as previously defined; (C₁₋₃)alkoxy means an alkoxy group having 1-3 carbon atoms, the alkyl moiety having the same meaning as previously defined; (C₁₋₂)alkoxy groups are preferred. (C₁₋₄)alkoxy means an alkoxy group having 1-4 carbon atoms, the alkyl moiety having the same meaning as previously defined. (C₁₋₃)alkoxy groups are preferred, (C₁₋₂)alkoxy groups being most preferred.

(C₂₋₄)alkenyl means a branched or unbranched alkenyl group having 2-4 carbon atoms, such as ethenyl, 2-propenyl, isobutenyl or 2-butenyl. (C₂₋₆)alkenyl means a branched or unbranched alkenyl group having 2-6 carbon atoms, such as ethenyl, 2-butenyl, and n-pentenyl, (C₂₋₄)alkenyl groups being most preferred.

(C₂₋₄)alkynyl means a branched or unbranched alkynyl group having 2-4 carbon atoms, such as ethynyl, 2-propynyl or 2-butynyl; (C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl. (C₂₋₄)alkynyl groups are preferred.

(C₃₋₆)cycloalkyl means a cycloalkyl group having 3-6 carbon atoms, being cyclopropyl, cyclobutyl, cyclopentyl or cyclohexyl; (C₃₋₇)cycloalkyl means a cycloalkyl group having 3-7 carbon atoms, being cyclopropyl, cyclobutyl, cyclopentyl, cyclohexyl or cycloheptyl; (C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; also preferred are piperidine, morpholine, pyrrolidine and piperazine; with the most preferred (C₂₋₆)heterocycloalkyl being pyrrolidine; the heterocycloalkyl group may be attached via a heteroatom if feasible; (C₃₋₇)heterocycloalkyl means a heterocycloalkyl group having 3-7 carbon atoms, preferably 3-5 carbon atoms, and one or two heteroatoms selected from N, O and/or S. Preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; and the heterocycloalkyl group may be attached via a heteroatom if feasible;

(C₃₋₇)cycloalkoxy means a cycloalkyl group having 3-7 carbon atoms, with the same meaning as previously defined, attached via a ring carbon atom to an exocyclic oxygen atom;

(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;

(C₁₋₅)heteroaryl means a substituted or unsubstituted aromatic group having 1-5 carbon atoms and 1-4 heteroatoms selected from N, O and/or S; the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, a more preferred (C₁₋₅)heteroaryl is pyrimidyl; (C₁₋₉)heteroaryl means a substituted or unsubstituted aromatic group having 1-9 carbon atoms and 1-4 heteroatoms selected from N, O and/or S; the (C₁₋₉)heteroaryl may optionally be substituted; preferred (C₁₋₉)heteroaryl groups are quinoline, isoquinoline and indole;

[(C₁₋₄)alkyl]amino means an amino group, monosubstituted with an alkyl group containing 1-4 carbon atoms having the same meaning as previously defined; preferred [(C₁₋₄)alkyl]amino group is methylamino; di[(C₁₋₄)alkyl]amino means an amino group, disubstituted with alkyl group(s), each containing 1-4 carbon atoms and having the same meaning as previously defined; preferred di[(C₁₋₄)alkyl]amino group is dimethylamino; (C₁₋₃)alkyl-C(O)—S—(C₁₋₃)alkyl means an alkyl-carbonyl-thio-alkyl group, each of the alkyl groups having 1 to 3 carbon atoms with the same meaning as previously defined; (C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; cyclohexenyl groups are most preferred; (C₂₋₆)heterocycloalkenyl means a heterocycloalkenyl group having 2-6 carbon atoms, preferably 3-5 carbon atoms; and 1 heteroatom selected from N, O and/or S; preferred (C₂₋₆)heterocycloalkenyl groups are oxycyclohexenyl and azacyclohexenyl group. When, in the definition of a substituent, is indicated that “all of the alkyl groups” of said substituent are optionally substituted, this also includes the alkyl moiety of an alkoxy group.

Depending on the ring or moiety formed, valency is preserved for indicated atoms; for example, nitrogen, if present in X or Y, may carry a hydrogen. The term “substituted” means that one or more hydrogens on the designated atom/atoms is/are replaced with a selection from the indicated group, provided that the designated atom's normal valency under the existing circumstances is not exceeded, and that the substitution results in a stable compound. Combinations of substituents and/or variable moieties are permissible only if such combinations result in stable compounds. “Stable compound” or “stable structure” is defined as a compound or structure that is sufficiently robust to survive isolation to a useful degree of purity from a reaction mixture, and formulation into a drug product containing an efficacious active pharmaceutical ingredient. The term “optionally substituted” means optional substitution with the specified groups, radicals or moieties.

BTK inhibitors of general formula (1) and synthesis thereof are described in EP2824099, WO2013161848, U.S. Pat. Nos. 8,450,335, 8,609,679, and WO2013063401. Preferred BTK inhibitors of general formula (1) are selected from the group consisting of compound 1 of table 1 shown below, compound 2 of table 1 shown below, and the compounds disclosed in tables 1-1 through 1-27 of EP2824099, more preferably compound 1 or 2 in table 1 shown below.

BTK inhibitors of general formula (2) and synthesis thereof are described in WO2013010869, US2017136014, and US2017095471. Preferred BTK inhibitors of general formula (2) are compounds 3, 4, 5, or 6 of table 1 shown below and the compounds disclosed on page 9 line 9 through page 11 line 10 of WO2013010869, preferably compounds 3, 4 (acalabrutinib), 5, or 6 of table 1.

BTK inhibitors of general formula (3) and synthesis thereof are described in WO2013067260. Preferred BTK inhibitors of general formula (3) have R26 as indicated for R⁷ on page 24 of WO2013067260. Preferred BTK inhibitors of general formula (3) are compound 7 of table 1 shown below and the compounds disclosed in tables 1 and 2 of WO2013067260, preferably compound 4 of table 1 shown below.

BTK inhibitors of general formula (4) and synthesis thereof are described in US2015352116. Preferred BTK inhibitors of general formula (4) are compound 8 of table 1 shown below, (R)-1-(3-(4-amino-3-(4-phenoxphenyl)-1H-pyrazolo[3,4-d]pyrimidin-1-yl)piperidin-1-yl)prop-2-en-1-one, 1-[(3R)-3-[4-amino-3-(4-phenoxyphenyI)-1H-pyrazolo[3,4-d]pyrimid in-1-yl]piperid in-1-yl]prop-2-en-1-one, (S)-1-(3-(4-amino-3-(4-phenoxphenyI)-1H-pyrazolo[3,4-d]pyrimidin-1-yl)piperidin-1-yl)prop-2-en-1-one, preferably compound 8 of table 1 shown below. Compound 8 is ibrutinib.

BTK inhibitors of general formula (5) and synthesis thereof are described in US2015125446, WO2013/081016, WO2011/152351, and US2017136014. Preferred BTK inhibitors of general formula (5) are compound 9 of table 1 shown below and the compounds disclosed in WO2013/081016, WO2011/152351, preferably compound 9 of table 1.

BTK inhibitors of general formula (6) and synthesis thereof are described in WO2012156334. Preferred BTK inhibitors of general formula (6) are compound 10 of table 1 shown below and the compounds disclosed in claim 8 of WO2012156334, preferably compound 10 of table 1.

BTK inhibitors of general formula (7) and synthesis thereof are described in US2016303130, US2017209462, US2016022684, WO2013191965, and WO2017023815. Preferred BTK inhibitors of general formula (7) are compounds 8 or 11 of table 1 shown below, (R)-1-(3-(4-amino-3-(4-phenoxyphenyI)-1H-pyrazolo[3,4-d]pyrimidin-1-yl)piperidin-1-yl)prop-2-en-1-one, 1-[(3R)-3-[4-amino-3-(4-phenoxyphenyl)-1H-pyrazolo[3,4-d]pyrimidin-1-yl]piperidin-1-yl]prop-2-en-1-one, (S)-1-(3-(4-amino-3-(4-phenoxphenyl)-1H-pyrazolo[3,4-d]pyrimidin-1-yl)piperidin-1-yl)prop-2-en-1-one, and the compounds disclosed in embodiment L starting on page 43 of WO2013191965, preferably compounds 8 or 11 of table 1.

BTK inhibitors of general formula (8) and synthesis thereof are described in WO2012170976. Preferred BTK inhibitors of general formula (8) are compound 12 of table 1 shown below, and the compounds disclosed in claim 9 of WO2012170976, preferably compound 12 of table 1 shown below.

BTK inhibitors of general formula (9) and synthesis thereof are described in

US2017/0136014. A preferred BTK inhibitors of general formula (9) is compound 13 of table 1 shown below.

BTK inhibitors of general formula (10) and synthesis thereof are described in WO2014173289. Preferred BTK inhibitors of general formula (10) are compounds 14 and 15 of table 1 shown below, and the compounds disclosed in table 3 in paragraph [0643] of WO2014173289, preferably compound 14 and 15 of table 1 shown below.

In preferred embodiments, the BTK inhibitor is chosen from table 1.

TABLE 1
preferred BTK inhibitors for use in the invention.

| Compound | | |
|---|---|---|
| number | Structure | Source |
| 1 | 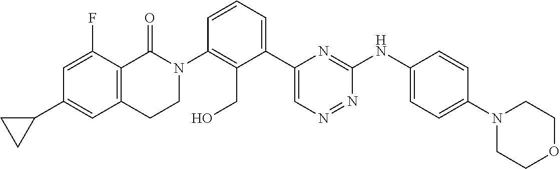 [SMILES: O=C1c2c(F)cc(C3CC3)cc2CCN1c1cccc(-c2cnnc(Nc3ccc(N4CCOCC4)cc3)n2)c1CO] | WO2013161848 |
| 2 | 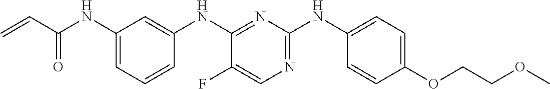 [SMILES: C=CC(=O)Nc1cccc(Nc2nc(Nc3ccc(OCCOC)cc3)ncc2F)c1] | WO2013063401 |
| 3 | 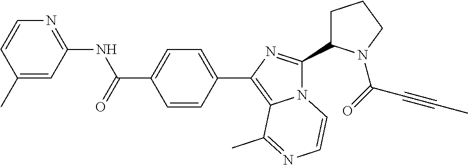 | WO2013010869 |
| 4 | 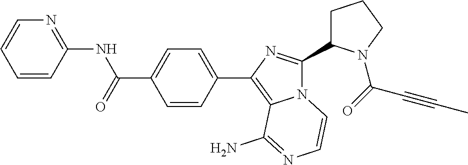 | US2017136014 |
| 5 | 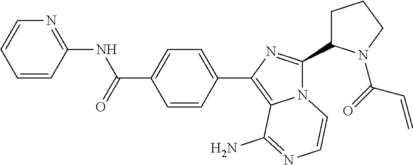 | US2017136014 |
| 6 | 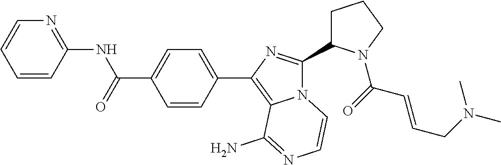 | US2017136014 |
| 7 | 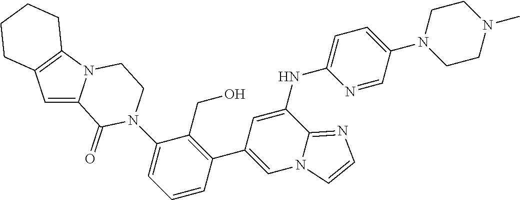 [SMILES: CN1CCN(c2ccc(Nc3cc(-c4cccc(N5CCn6c(cc7c6CCCC7)C5=O)c4CO)cn4ccnc34)nc2)CC1] | WO2013067260 |
| 8 | 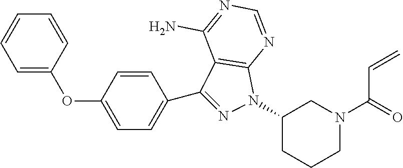 [SMILES: C=CC(=O)N1CCC[C@H](n2nc(-c3ccc(Oc4ccccc4)cc3)c3c(N)ncnc32)C1] | US2015352116 |
| 9 | 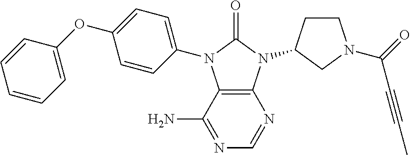 [SMILES: CC#CC(=O)N1CC[C@@H](n2c(=O)n(-c3ccc(Oc4ccccc4)cc3)c3c(N)ncnc32)C1] | US2015125446 |
| 10 | 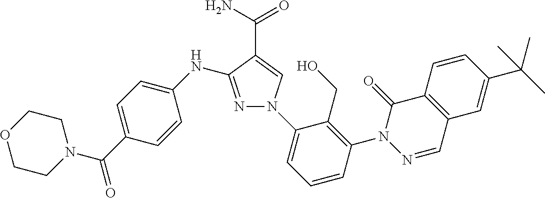 [SMILES: CC(C)(C)c1ccc2c(=O)n(-c3cccc(-n4cc(C(N)=O)c(Nc5ccc(C(=O)N6CCOCC6)cc5)n4)c3CO)ncc2c1] | WO2012156334 |
| 11 | 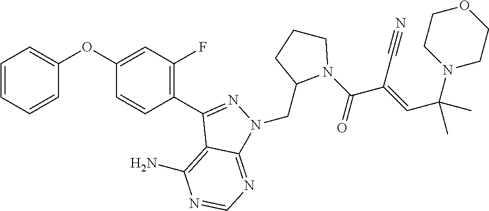 [SMILES: CC(C)(/C=C(\C#N)C(=O)N1CCCC1Cn1nc(-c2ccc(Oc3ccccc3)cc2F)c2c(N)ncnc21)N1CCOCC1] | WO2013191965 |
| 12 | 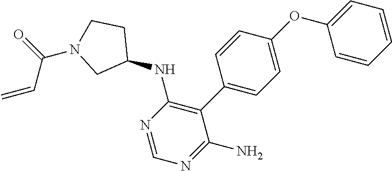 [SMILES: C=CC(=O)N1CC[C@@H](Nc2ncnc(N)c2-c2ccc(Oc3ccccc3)cc2)C1] | WO2012170976 |
| 13 | 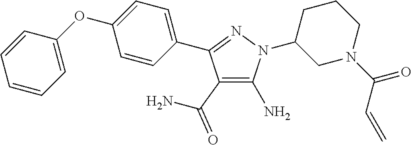 [SMILES: C=CC(=O)N1CCCC(n2nc(-c3ccc(Oc4ccccc4)cc3)c(C(N)=O)c2N)C1] | US2017/0136014 |
| 14 | 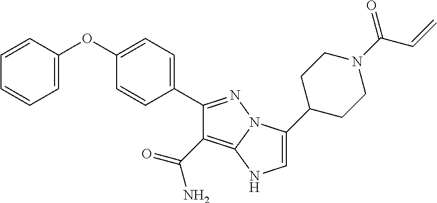 [SMILES: C=CC(=O)N1CCC(c2c[nH]c3c(C(N)=O)c(-c4ccc(Oc5ccccc5)cc4)nn23)CC1] | WO2014173289 |
| 15 | 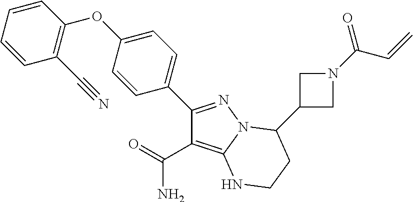 [SMILES: C=CC(=O)N1CC(C2CCNc3c(C(N)=O)c(-c4ccc(Oc5ccccc5C#N)cc4)nn32)C1] | WO2014173289 |

In some embodiments, the BTK inhibitor is selected from ibrutinib, zanabrutinib (BGB-3111), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In preferred embodiments, the BTK inhibitor is ibrutinib, zanabrutinib (BGB-3111), or acalabrutinib (compound 4 of table 1), of which ibrutinib is most preferred.

### The Bcl-2 Inhibitor

The term “Bcl-2 inhibitor” as used herein, refers to a compound that selectively targets, decreases, or inhibits at least one activity of Bcl-2. The B-cell lymphoma 2 (Bcl-2) family of proteins are key regulators of the mitochondrial (also called “intrinsic”) pathway of apoptosis. See, Denial, N. N. and Korsmeyer, S. J. Cell (2004) 116, 205-219. Misregulation of Bcl-2 is implicated in a wide variety of cancers, particularly hematological cancers such as follicular lymphoma, diffuse large cell lymphoma, and chronic lymphocytic leukemia. Adams, J. M. and Cory, S. Science (1998) 281, 1322-1326. As used herein “Bcl-2” refers to B-cell lymphoma 2, an anti-apoptotic protein that has been implicated in numerous types of cancers including chronic lymphocytic leukemia, melanoma, breast, prostate and lung carcinomas. Targeted and selective Bcl-2 inhibitors exist and include oblimersen, navitoclax, and venetoclax. In preferred embodiments, the Bcl-2 inhibitor is an antisense oligonucleotide that targets Bcl-2, or a small molecule. In more preferred embodiments the Bcl-2 inhibitor is a small molecule. In other more preferred embodiments the Bcl-2 inhibitor is an antisense oligonucleotide that targets Bcl-2. In preferred embodiments the Bcl-2 inhibitor is not an antibody.

### Antisense Oligonucleotide Bcl-2 Inhibitor

Antisense oligonucleotides that target Bcl-2 can reduce Bcl-2 activity and thus act as a Bcl-2 inhibitor. Preferred examples are oblimersen and PNT2258. In more preferred embodiments, an antisense oligonucleotide Bcl-2 inhibitor is oblimersen. In other more preferred embodiments, an antisense oligonucleotide Bcl-2 inhibitor is PNT2258. In preferred embodiments, an antisense oligonucleotide Bcl-2 inhibitor is encapsulated in a liposomal formulation, more preferably in smarticles as described in Tolcher et al., 2013, DOI: 10.1007/s00280-013-2361-0.

An antisense DNA or RNA strand is non-coding and complementary to the coding strand (which is the template for producing respectively RNA or protein). An antisense drug is a short sequence of RNA that hybridizes with and inactivates mRNA, preventing the protein from being formed. Human lymphoma cell proliferation (with t(14;18) translocation) is inhibited by antisense RNA such as oblimersen targeted at the start codon region of Bcl-2 mRNA. In vitro studies led to the identification of oblimersen (also known as Genasense), which is complementary to the first 6 codons of Bcl-2 mRNA. Antisense oligonucleotides showed successful results in Phase I/II trials for lymphoma. Preferred antisense oligonucleotide inhibitors are discussed in Dias and Stein (2002, Eur. J. Pharm. Biopharm.) DOI: 10.1016/S0939-6411(02)00060-7. PNT2258 is a liposomal formulation of an antisense oligonucleotide Bcl-2 inhibitor that has been developed by ProNAi Therapeutics, Inc. (see e.g. Ebrahim et al., 2016, DOI: 10.18632/oncotarget.9872 or Tolcher et al, cited above).

### Small Molecule Bcl-2 Inhibitor

Examples of small molecules that inhibit Bcl-2 are ABT-737 and navitoclax (ABT-263) and venetoclax (ABT-199). Abbott Laboratories developed ABT-737 and navitoclax, which are part of a group of BH3 mimetic small molecule inhibitors (SMI) that target the Bcl-2 family proteins, but not A1 or Mcl-1. They are superior to previous Bcl-2 inhibitors given their higher affinity for Bcl-2, Bcl-xL and Bcl-w. In vitro studies showed that primary cells from patients with B-cell malignancies are sensitive to ABT-737, which is a preferred Bcl-2 inhibitor. ABT-737 does not directly induce apoptosis; it enhances the effects of apoptotic signals and causes single-agent-mechanism-based killing of cells in small-cell lung carcinoma and lymphoma lines. In animal models, it improves survival, causes tumor regression and cures a high percentage of mice. In preclinical studies utilizing patient xenografts, ABT-737 shows efficacy for treating lymphoma and other blood cancers.

Abbvie developed the highly selective inhibitor venetoclax (ABT-199), which inhibits Bcl-2, but not Bcl-xL or Bcl-w. Clinical trials studied the effects of venetoclax, a BH3-mimetic drug designed to block the function of the Bcl-2 protein, on patients with chronic lymphocytic leukemia (CLL) (Roberts et al., 2016, doi:10.1056/NEJMoal 513257). Good responses have been reported and thrombocytopenia was not observed, rendering venetoclax a highly preferred Bcl-2 inhibitor. It was approved by the US FDA in April 2016 as a second-line treatment for CLL associated with 17-p deletion. In June 2018, the FDA broadened the approval for anyone with CLL or small lymphocytic lymphoma, with or without 17p deletion, still as a second-line treatment.

In preferred embodiments, a Bcl-2 inhibitor is of general formula B1

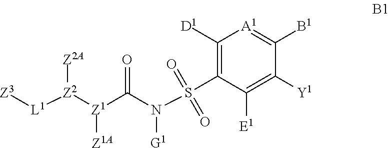

wherein definitions for variable moieties (as represented by letters with numerical and/or alphabetical superscripts) are as provided in claim 1 of WO2011149492. These compounds and their synthesis are known from WO2011149492. Preferred compounds of general formula B1 have variable moieties as defined on page 76, line 19 through page 85, line 26 of WO2011149492, more preferably as defined on page 84, line 28 through page 93, line 2.

In preferred embodiments, a Bcl-2 inhibitor of general formula B1 is of general formula B2

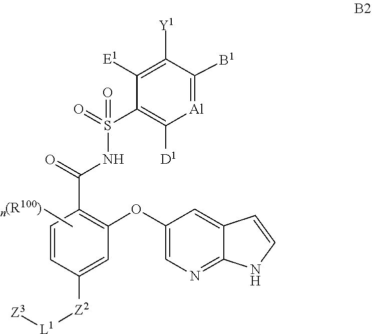

wherein definitions for variable moieties (as represented by letters with numerical and/or alphabetical superscripts) are as provided in [0007] of EP2435432B1, more preferably they are as provided in claim 2 of EP2435432B1. Such Bcl-2 inhibitors and their preparation are known from EP2435432B1. Preferred compounds of general formula B2 are the compounds named in [0008] of EP2435432B1, or pharmaceutically acceptable salts thereof; more preferred compounds of general formula B2 are the compounds named in [0009] through [0028] of EP2435432B1, or pharmaceutically acceptable salts thereof; most preferred compounds of general formula B2 are 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide or a therapeutically acceptable salt thereof and 4-(4-{[2-(4-chlorophenyl)-4,4-dimethylcyclohex-1-en-1-yl]methyl}piperazin-1-yl)-N-[(4-{[(trans-4-hydroxy-4-methylcyclohexyl)methyl]amino}-3-nitrophenyl)sulfonyl]-2-(1H-pyrrolo[2,3-b]pyrid in-5-yloxy)benzamide or a therapeutically acceptable salt thereof. In preferred embodiments a compound of general formula B2 is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyDamino]phenyl}sulfonyl)-2-(1H-pyrrolo[2,3-b]pyridin-5-yloxy)benzamide or a therapeutically acceptable salt thereof. In preferred embodiments a compound of general formula B2 is 4-(4-{[2-(4-chlorophenyI)-4,4-dimethylcyclohex-1-en-1-yl]methyl}piperazin-1-yl)-N-[(4-{[(trans-4-hydroxy-4-methylcyclohexyl)methyl]amino}-3-nitrophenyl)sulfonyl]-2-(1H-pyrrolo[2,3-b]pyrid in-5-yloxy)benzamide or a therapeutically acceptable salt thereof.

In preferred embodiments, the Bcl-2 inhibitor is ABT-199 (venetoclax), ABT-737, ABT-263 (navitoclax), or PNT2258. In a more preferred embodiment, the Bcl-2 inhibitor is venetoclax or PNT2258. In other preferred embodiments, the Bcl-2 inhibitor is selected from the group consisting of 4-(4-{[2-(4-Chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]-benzamide (navitoclax or ABT-263); tetrocarcin A; antimycin; gossypol (preferably (−)-gossypol acetic acid, known as AT101); obatoclax (preferably obatoclax is its mesylate); ethyl 2-amino-6-bromo-4-(1-cyano-2-ethoxy-2-oxoethyl)-4H-chromene-3-carboxylate (HA 14-1); oblimersen; a Bak BH3 peptide; 4-[4-[(4′-chloro[1,T-biphenyl]-2-yl)methyl]-1-piperazinyl]A/-[[4-[[(1R)-3-(dimethylamino)-1-[(phenylthio)methyl]propyl]amino]-3-nitrophenyl]sulfonyl]benzamide (ABT-737); 2-((1 Hpyrrolo[2,3-b]pyridin-5-yl)oxy)-4-(4-((4(4′-chloro-5,5-dimethyl-3,4,5,6-tetrahydro-[1,T-biphenyl]-2-yl)methyl)piperazin-1-yl)-N-((3-nitro-4-(((tetrahydro-2H-pyran-4-yl)methyl)amino)phenyl)sulfonyl)benzamide (venetoclax); and S55746 (BCL201), or pharamceutically acceptable salts thereof. S55746 is known from clinical trials for patients with chronic lymphocytic leukemia (CLL), B-cell non-Hodgkin's lymphoma, or multiple myeloma, (see e.g. clinicaltrials.gov/ct2/show/NCT02920697).

The structural formula of venetoclax is C₄₅H₅₀CIN₇O₇S (4-(4-{[2-(2-(4-chlorophenyl)-4,4-dimethylcyclohex-2H-pyrrolo[2,3-b]pyridine-5-yloxy)benzamide)). Venetoclax binds to two hydrophobic pockets of Bcl-2, and creates a hydrogen bond between the azaindole nitrogen of venetoclax and Asp103 and Arg107 of BLC-2. This electrostatic interaction prevents the inhibitory action of the Bcl-2 against BAK and BAX, so these proteins can initiate the intrinsic pathway of apoptosis (Scheffold et al., 2018, supra; Moia et al., 2018, supra). Despite this drug selectively binds with high affinity to Bcl-2, it also binds to the pro-apoptotic BCL-XL and BCL-W with significantly lower affinity whereas no activity against the pro-apoptotic protein MCL-1 has been reported (Scheffold et al., 2018, supra; Moia et al., 2018, supra).

Structures of preferred Bcl-2 are shown below. These compounds are known in the art.

| Number | Name | Structure |
|---|---|---|
| 1 | Venetoclax (ABT-199) | 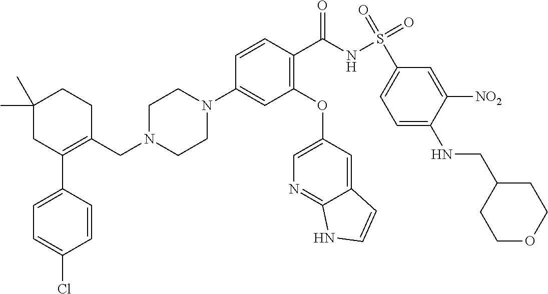 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1] |
| 2 | Navitoclax (ABT-263) | 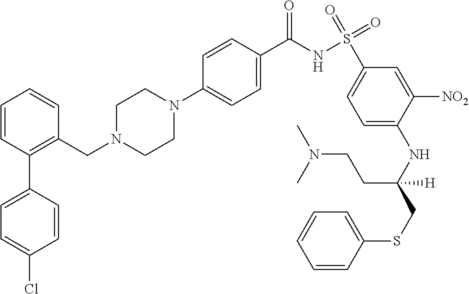 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]] |
| 3 | Obatoclax | 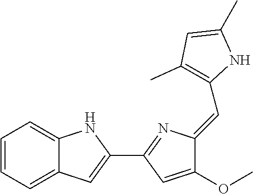 [SMILES: COC1=CC(c2cc3ccccc3[nH]2)=N/C1=C\c1[nH]c(C)cc1C] |
| 4 | HA 14-1 | 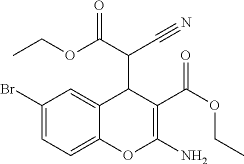 [SMILES: CCOC(=O)C1=C(N)Oc2ccc(Br)cc2C1C(C#N)C(=O)OCC] |
| 5 | ABT-737 | 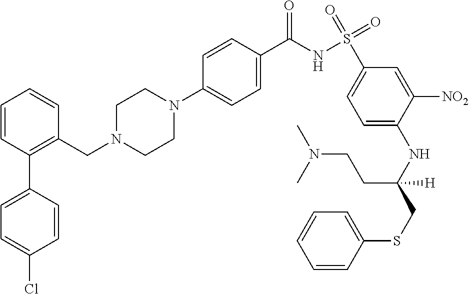 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]] |
| 6 | A-1331852 | 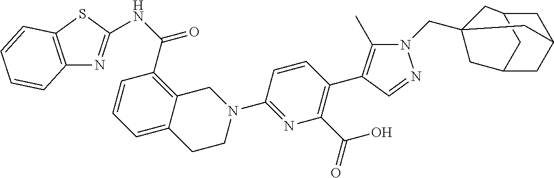 [SMILES: Cc1c(-c2ccc(N3CCc4cccc(C(=O)Nc5nc6ccccc6s5)c4C3)nc2C(=O)O)cnn1CC12CC3CC(CC(C3)C1)C2] |
| 7 | TW-37 | 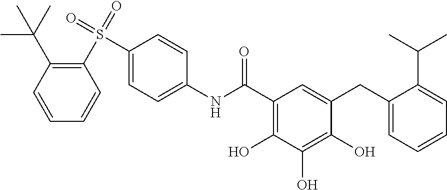 [SMILES: CC(C)c1ccccc1Cc1cc(C(=O)Nc2ccc(S(=O)(=O)c3ccccc3C(C)(C)C)cc2)c(O)c(O)c1O] |
| 8 | Gossypol | 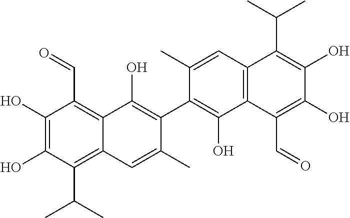 [SMILES: Cc1cc2c(C(C)C)c(O)c(O)c(C=O)c2c(O)c1-c1c(C)cc2c(C(C)C)c(O)c(O)c(C=O)c2c1O] |
| 9 | Sabutoclax | 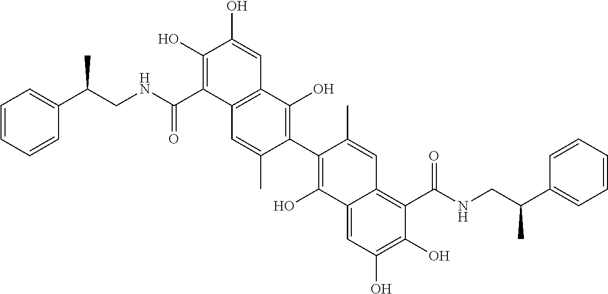 [SMILES: Cc1cc2c(C(=O)NC[C@H](C)c3ccccc3)c(O)c(O)cc2c(O)c1-c1c(C)cc2c(C(=O)NC[C@H](C)c3ccccc3)c(O)c(O)cc2c1O] |
| 10 | S55746 | 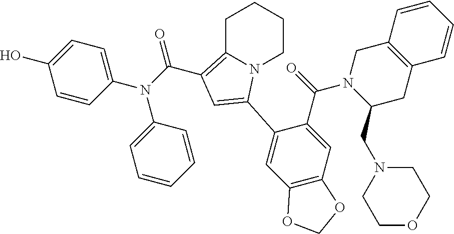 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1] |
| 11 | Gambogic acid | 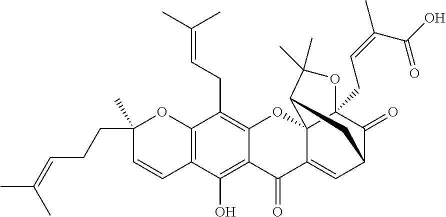 [SMILES: CC(C)=CCC[C@]1(C)C=Cc2c(O)c3c(c(CC=C(C)C)c2O1)O[C@]12C(=C[C@@H]4C[C@H]1C(C)(C)O[C@@]2(C/C=C(/C)C(=O)O)C4=O)C3=O] |

In preferred embodiments, the Bcl-2 inhibitor is chosen from compounds 1-11 in the table above, more preferably it is chosen from compounds 1, 2, 3, 4, 5, 6, 7, 9, and 10, even more preferably it is chosen from compounds 1, 2, 3, 4, 5, 6, 7, and 10, more preferably still it is chosen from compounds 1, 2, 3, 4, and 5, most preferably from compounds 1, 2, and 5.

In preferred embodiments, the Bcl-2 inhibitor or a composition comprising such a Bcl-2 inhibitor is administered for at least one cycle of 5 days; even more preferably such a Bcl-2 inhibitor is a small molecule, most preferably venetoclax.

### The Pharmaceutical Composition

In another aspect, the invention relates to a pharmaceutical composition comprising at least one of an anti-CCR7 antibody (or antigen-binding fragment thereof) as herein defined, a BTK inhibitor as herein defined and a Bcl-2 inhibitor as herein defined, for a use in accordance with the invention. The pharmaceutical composition preferably at least comprises at least one of the BTK inhibitor, the Bcl-2 inhibitor and the anti-CCR7 antibody or a pharmaceutical derivative or prodrug of these active ingredients, together with a pharmaceutically acceptable carrier, adjuvant, or vehicle, for administration to a subject. Said pharmaceutical composition can be used in the methods of treatment described herein below by administration of an effective amount of the composition to a subject in need thereof. The term “subject” is used interchangeably with the term “recipient” herein, and as used herein, refers to all animals classified as mammals and includes, but is not restricted to, primates and humans. The subject is preferably a male or female human of any age or race. The treatment of the patient includes treatment in the first line or second line, or third line.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g.

“Handbook of Pharmaceutical Excipients”, Rowe et al eds. 7^{th} edition, 2012, www.pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, PLURONICS™ or polyethylene glycol (PEG).

The antibodies of the invention may be formulated in the same pharmaceutical composition as the BTK inhibitor and the Bcl-2 inhibitor or each of the actives may be formulated in its own pharmaceutical composition. The preferred route of administration will determine the preferred formulation for each of the actives. As parenteral is the preferred route of administration for the antibody, while the preferred route of administration for at least some of the BTK- and Bcl-2 inhibitors is oral, the antibody preferably is formulated in a different pharmaceutical composition than the inhibitors. Administration of the antibody and at least one of the BTK- and Bcl-2 inhibitors can be concurrent or sequential, and may be effective in either order.

Supplementary active compounds can also be incorporated into the pharmaceutical compositions of the invention. Thus, in a particular embodiment, the pharmaceutical composition of the invention may also contain more than one active compound as necessary for the particular indication being treated, preferably those with complementary activities that do not adversely affect each other. For example, it may be desirable to further provide a chemotherapeutic agent, a cytokine, an analgesic agent, or an immunomodulating agent, e.g. an immunosuppressive agent or an immunostimulating agent. The effective amount of such other active agents depends, among other things, on the amount of antibody of the invention present in the pharmaceutical composition, the type of disease or disorder or treatment, etc.

In an embodiment, the antibody of the invention is prepared with carriers that will protect said compound against rapid elimination from the body, such as a controlled release formulation, including implants and microencapsulated delivery systems, e.g. liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811 or US 2011305751, incorporated herein by reference.

The administration route of the antibody (or fragment thereof) of the invention can be oral, parenteral, by inhalation or topical. The term “parenteral” as used herein includes intravenous, intra-arterial, intralymphatic, intraperitoneal, intramuscular, subcutaneous, rectal or vaginal administration. The intravenous forms of parenteral administration are preferred. By “systemic administration” is meant oral, intravenous, intraperitoneal and intramuscular administration. The amount of an antibody required for therapeutic or prophylactic effect will, of course, vary with the antibody chosen, the nature and severity of the condition being treated and the patient. In addition, the antibody may suitably be administered by pulse infusion, e.g., with declining doses of the antibody. Preferably the dosing is given by injections, most preferably intravenous or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminum monostearate and gelatin.

Sterile injectable solutions can be prepared by incorporating the active compound (e.g., a BTK inhibitor and/or a Bcl-2 inhibitor or anti-CCR7 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV) or subcutaneous (SC). Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, www.pharmpress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (antibody of the invention) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, an effective administered amount of an antibody of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.001 to 1,000 mg/kg body weight/day, preferably about 0.01 to about 100 mg/kg body weight/day, most preferably from about 0.05 to 10 mg/kg body weight/day. More specifically, for use in accordance with the invention, the anti-CCR7 antibodies are preferably administered at a dosage of 1-1000, 2-500, 5-200, 10-100, 20-50 or 25-35 mg/kg body weight/day, preferably administered in doses every 1, 2, 4, 7, 14 or 28 days.

Aside from administration of antibodies to the patient, the present application contemplates administration of antibodies by gene therapy. WO 96/07321 relates the use of gene therapy to generate intracellular antibodies.

The pharmaceutical compositions can be included in a container, pack, or dispenser together with instructions for administration.

The antibodies, in and pharmaceutical compositions of this invention may be used with other drugs to provide a combination therapy. The other drugs may form part of the same composition or be provided as a separate composition for administration at the same time or at different time.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5 or 10% of the value.

All patent and literature references cited in the present specification are hereby incorporated by reference in their entirety.

The present invention is further described by the following examples, which should not be construed as limiting the scope of the invention.

### DESCRIPTION OF THE FIGURES

FIG. 1. CCR7 expression is maintained in patients treated with BTK inhibitors. CCR7 expression is slightly diminished (ibrutinib) or unchanged (acalabrutinib or zanabrutinib) in CLL patients treated with BTK inhibitors. Expression of CCR7 and CD20 was determined in primary CLL cells from peripheral blood samples obtained from naïve patients (n=125), patients receiving ibrutinib treatment (OT, n=44), patients with relapsed/refractory disease to ibrutinib (RR, n=16), patients receiving acalabrutinib treatments (ACALA, n=5), or patients receiving zanabrutinib treatment (ZANA, n=4). A) Expression of surface CCR7 (or CD20) was analyzed in terms of relative median intensity of fluorescence (RMIF, relative to an irrelevant isotype control). B) Expression of surface CCR7 (or CD20) was analyzed in terms of proportion of malignant cells expressing the receptor.

FIG. 2. Expression of CCR7 in malignant cells is high irrespective of current or previous treatments.

A) The graphs show CCR7 expression from one representative naïve (untreated) CLL patient, one representative CLL patient receiving ibrutinib (OT), one representative CLL patient who failed to ibrutinib (RR), and one representative CLL patient receiving venetoclax. In each patient, CCR7 expression is disclosed as frequency histograms compared to a matched irrelevant control. Marker regions were placed on base to these irrelevant controls. Each histogram shows the intensity of fluorescence for cells labelled with anti-CCR7-PE. The proportion of CCR7-positive cells (within the marker) is also shown. B) Changes in the expression of CCR7 and CD20 in four CLL patients that started treatment with the BTK inhibitor ibrutinib. For each patient, the expression of CCR7 (determined as RMIF) before (N) and after administration (Y) of ibrutinib is shown.

FIG. 3. CCR7 expression is maintained in patients treated with Bcl-2 inhibitors.

CCR7 expression is maintained in essentially all CLL cells from patients treated with venetoclax. Expression of CCR7 and CD20 was determined in CLL cells from peripheral blood samples obtained from naïve patients (n=125) or patients receiving venetoclax treatment (OT, n=11). A) Expression of surface CCR7 (or CD20) was analyzed in terms of relative median intensity of fluorescence (RMIF, relative to an irrelevant isotype control). B) Expression of surface CCR7 and CD20 was analyzed in terms of proportion of malignant cells expressing the receptor.

FIG. 4. Treatment (in vivo) with ibrutinib does not neutralize migration mediated by CCR7 in CLL cells.

A) Comparative analysis of migration indices (% of input) in CLL cells obtained from naïve untreated patients (N, n=7, black bars) versus cells obtained from CLL patients receiving ibrutinib (OT, n=10, grey bars). Migration was achieved with exposure of CLL cells to CCR7 ligands, CCL19 (1 μg/ml) or CCL21 (1 μg/ml). Migration was tested in chemotaxis assays where cells were placed on nude transwell chambers. Assays were conducted for 4 hours at 37° C. Spontaneous migration, not mediated by a chemotactic stimulus was considered as basal migration (in this point, no chemokine was added). B) Comparative analysis of migration indices (% of input) in CLL cells from naïve untreated patients (n=7) that were exposed for one hour to different concentrations of ibrutinib at final concentrations (0-vehilce/DMSO; 0.01, 0.1, 1, 10 μM) prior to exposure to CCR7 ligands CCL19 (1 μg/ml) or CCL21 (1 μg/ml). Migration was tested in chemotaxis assays where cells were placed on nude transwell chambers. Assays were conducted for 4 hours at 37° C. (Ibrutinib was present during all the migration time). Spontaneous migration, not mediated by a chemotactic stimulus, was considered as basal migration (in this point, no chemokine was added). As positive controls, cells without ibrutinib exposure where used (black bars) In A and B, bars represent mean±standard error of the mean (SEM). ns, not significant; *, p<0.05, **, p<0.01.

FIG. 5: Treatment (in vitro) with ibrutinib at 0.1 μM for 24 h does not neutralize migration mediated by CCR7 in CLL cells.

Comparative analysis of migration indices (% of input) in CLL cells obtained from naïve untreated patients (n=6) that where incubated for 24 h with ibrutinib at a final concentration of 0 (vehicle/DMSO) or 0.1 μM prior to exposure to CCR7 ligands CCL19 (1 μg/ml) or CCL21 (1 μg/ml). Migration was tested in chemotaxis assays where cells were placed on nude transwell chambers. Migration assays were conducted for 4 hours at 37° C. Spontaneous migration, not mediated by a chemotactic stimulus, was considered as basal migration (in this point, no chemokine was added). As positive controls, cells without ibrutinib exposure where used (black bars). Bars represent mean±standard error of the mean (SEM). ns, not significant; *, p<0.05.

FIG. 6. Treatment with an anti-CCR7 antibody CAP-100 neutralizes CCR7-mediated migration of CLL cells from patients treated with BTK inhibitors or Bcl-2 inhibitors.

The specific blocking of CCR7-ligand interactions, expressed as a reduction of % of migrating input cells is shown for CLL cells obtained from a patient receiving venetoclax (A) or for CLL cells obtained from a patient receiving ibrutinib (B). CLL cells were incubated with an anti-CCR7 antibody (CAP-100) at different final concentrations (100, 10, 1, 0.1, 0.01, 0 μg/ml). Subsequently, cells were placed on transwell chambers and exposed to the chemokines CCL19 (white bars) and CCL21 (grey bars) at 1 μg/ml. Migration assays on naked transwell chambers were conducted for 4 hours at 37° C. Basal migration is the spontaneous migration, not mediated by a chemotactic stimulus—in this point no chemokine or mAb is added. Maximum migration is observed when chemokines are added but no CAP-100 is present (point 0).

FIG. 7. Effect of the combination of ibrutinib with anti-CCR7 antibody CAP-100 on the migration induced by CCR7 ligands.

Comparative analysis of migration indices (% of input) in CLL cells obtained from naïve untreated patients (n=5) that were incubated with anti-CCR7 antibody (CAP-100; 10 μg/ml), with ibrutinib (0.1 μM), or with a combination of both compounds (CAP-100 at 10 μg/ml; ibrutinib at 0.1 μM) before performing chemotaxis assays towards CCR7 ligands [CCL19 (1 μg/ml) or CCL21 (1 μg/ml); in nude transwell chambers]. Spontaneous migration, not mediated by a chemotactic stimulus was considered as basal migration (in this point, no chemokine was added; white bar). As positive controls, cells without anti-CCR7 antibody or ibrutinib pre-incubation where used (black bars). Bars represent mean±standard error of the mean (SEM). ns, not significant; *, p<0.05.

FIG. 8. CCR7 expression in CLL cells from patients under treatment with BTK or Bcl-2 inhibitors can effectively induce cell death upon binding of an anti-CCR7 antibody.

ADCC activity was assayed in one patient receiving treatment with ibrutinib (A), in one patient receiving treatment with zanabrutinib (B), in one patient receiving venetoclax (C), and in one patient with relapsed/refractory disease to ibrutinib (D). In all cases, CLL cells were incubated in the presence of an irrelevant matched isotype control, or with anti-CD20 antibody (rituximab, RTX), or with anti-CCR7 antibody (CAP-100). Antibodies were tested at different final concentrations (0, 0.01, 0.1, 1, 10, 100 μg/ml). To perform ADCC, isolated PBMCs (peripheral blood mononuclear cells) were used as effector cells at a fixed E:T ratio of 10:1. The percentage of CLL cells killed by ADCC was determined by flow cytometry based on the incorporation of 7-aminoactinomycinD (7-AAD). The proportion of specific lysis induced by the antibodies is shown.

### EXAMPLES

### Introduction

The chemokine receptor CCR7 controls migration of certain immune cell subsets to the lymph nodes, where, in addition it contributes to immune cells organization and activation (Legler et al., Int J Biochem Cell Biol. 2014; 54:78-82). In agreement with a lymphoid origin, several blood cancers with lymph node involvement express CCR7 (LOpez-Giral et al., J Leukoc Biol. 2004; 76(2):462-71). In this diseases CCR7 expression correlates with bulky lymphadenopathy, aggressive disease, and short survival (Legler et al., 2014; supra). Specifically, in B-cell malignancies, CCR7 expression contributes to extend tumor cell residency in the LN as well as to guide tumor cells towards niches were pro-tumor cues can be achieved (Rehm et al., Blood, 2011; 118(4):1020-33).

Recently, some reports disclosed that ibrutinib treatment of CLL cells resulted in a down-regulation in surface CCR7 which, in addition, translated to a subsequent impairment in CCR7-mediated migration and CCR7-mediated adhesion (Patrussi et al., 2015; supra 75(19):4153-63; de Rooij et al., 2012; supra). As result of these works, it was proposed that one off-target effect mediated by ibrutinib was to decrease lymph node migration, partially caused by a restoration of increased surface CCR7 in CLL cells to levels normally expressed in B-cells. Similarly, a positive feedback on expression profiles has been reported for CCR7 and Bcl-2 (Kim et al., 2005; supra).

On the basis of the described findings, a combination of a BTK inhibitor, and/or a Bcl-2 inhibitor with an anti-CCR7 antibody would not be expected to improve the therapeutic inhibition of migration of malignant cells to the lymph nodes (or other SLOs). Moreover, based on the previously reported loss of CCR7 expression, a therapeutic approach aiming to target CCR7 to kill tumor cells would be precluded and would not be expected to improve individual therapeutic efficacy of BTK or Bcl-2 inhibitors.

Herein the inventors document the impact of BTK or Bcl-2 inhibitors on CCR7 expression in CLL patients and perform several approaches to investigate whether these compounds could negatively impact on CCR7-triggered functions (such as migration) as well as whether these compounds could prevent an effective target cell killing mediated by anti-CCR7 antibodies as a result of a lack of CCR7 expression on target cell surface.

### Materials and methods

### Samples

Leukemic cells from patients were isolated from the freshly donated peripheral blood using ficoll-paque plus density gradient centrifugation (Amersham Biosciences). Isolated cells were maintained for short-term cultures in RPMI 1640 media supplemented with 10% heat-inactivated fetal bovine serum (FBS), 2 mM L-glutamine and 100 U/mL penicillin/100 μg/mL streptomycin at 37° C. in 5% CO2. Normal peripheral blood mononuclear cells (PBMCs) were obtained from adult blood buffy coats after ficoll gradient and maintained in complete medium as described above. In all cases, patients and healthy donor signed an informed consent in accordance to the Declaration of Helsinki. Experimental procedures were approved by the Institutional review Board of the Hospital de la Princesa.

Three types of patient samples (n=196) were included in this study:
1) Naïve patients (i.e., patients not treated before with either a BTK inhibitor or a Bcl-2 inhibitor).
2) On ibrutinib (or other BTK inhibitor) treatment patients.
3) Ibrutinib R/R patients.

### CCR7 Expression

Flow cytometry analysis of CCR7 and CD20 expression in CLL cells was performed with a four-colour panel of monoclonal antibodies: CD19-APC-H7 (BD Biosciences), CD3-FITC (BD Biosciences), CD5-APC (BD Biosciences) and either CCR7-PE (R&D Systems) or CD20-PE (R&D

Systems). Studies were conducted on the CD19⁺ CD3⁻CD5⁺ CLL population. An appropriate matched isotype control (IC) conjugated to PE was included (R&D Systems). To determine receptor expression, 10⁶ cells (in ˜50 μl of PB) were incubated with the antibodies for 15-20 minutes at room temperature (RT). Cells were lysed with BD FACS™ Lysing Solution (BD Biosciences) for 10 minutes at RT and centrifugated at 1.800 rpm for 2 minutes. Then, cells were washed with 2m1 of Dulbecco's Phosphate Buffered Saline (PBS) (Lonza) and, finally centrifugated at 1.800 rpm for 2 minutes. Data acquisition was performed on a BD FACSCanto™ 11 Flow Cytometer (BD Biosciences). A minimum of 10.000 CLL cells were analysed using the BD FACSDIVA™ Software. Results are expressed both as a percentage of CCR7 and CD20 positive cells [% receptor−% control] and relative median fluorescence intensity (RMFI) of CCR7 and CD20 expression compared to the IC [MIF(receptor)/MIF(control)].

### Chemotaxis Assay (Migration)

PBMCs were isolated by centrifugation on Biocoll Separating Solution density gradient centrifugation (Merck Millipore). PBMC were washed twice with saline solution (Fresenius) and centrifugated at 1200 for 10 minutes. Cells were suspended in RPMI 1640 media (GIBCO) supplemented with 0.1% bovine serum albumin (BSA) at a concentration of 5×10⁶ cells/ml. When indicated, and prior to the chemotaxis assay, cells were pre-incubated with the followings reagents: a) Ibrutinib, 1 hour at RT; at different final concentrations; b) Ibrutinib, 24 hours at 37° C.; at different final concentrations; b) anti-CCR7 mAb, 0.5 h at RT. Chemotaxis was performed in Transwell chambers (6.5-mm diameter, 10-μm thickens, 5-μm diameter pore size, Costar). Only samples from patients with >90% of tumour cells were included. A total of 5×10⁵ cells suspended in RPMI-1640, 0.1% BSA were loaded in the upper chamber and chemokines CCL19 or CCL21 (1 μg/ml, Peprotech) were added to the lower well. After 4 hours at 37° C. 5% CO, cells in the lower chamber were collected, and stained with anti-CD3-PE (BD Biosciences) and anti-CD5-APC (BD Biosciencies). The migrated cells were counted for 60 seconds in a BD FACSCanto™ II flow cytometer. The percentage of migrated cells (% of input) was calculated according to the following formula: 100×(number of cells in the lower chamber/number of cells loaded in the upper chamber). Once the % of input was calculated, the % of inhibition was estimated by means the following formula: % of inhibition=[(% input without mAb−% input with mAb)×100]/[% input without mAb]. In addition, results are shown as the % of input relative to the maximum effect mediated by each chemokines (CK) which was calculated according to: %input rel. to CK=100×(% input/% input with CK).

### Antibody-Dependent Cell-Mediated Cytotoxicity (ADCC)

ADCC assays were performed as described in SR-HPM-1026. Briefly: target tumour cells were incubated with media alone (RPMI+10% FBS) or in the presence of IC, rituximab, alemtuzumab, or CAP-100 antibodies, at different final concentrations, at 37° C. for 30 min. Unbound antibody was washed off (1800 rpm, 2 minutes/twice) and the cells plated at 10⁵ cells/well in p96 U-bottom plates. Human PBMCs from healthy donors were obtained by ficoll density gradient centrifugation. Effector cells were labelled with calcein-UV Cell Tracker (Invitrogen) according to the manufacturer's protocol, and stimulated with recombinant human IL-2 (500 UI/ml, StemCell Technologies). Different effector:target (E:T) ratios were used [10:1 for dose-response assays, and (5:1, 25:1, 50:1) for E:T assays]. After 6 hours, cells were stained with 7AAD, CD3-PE and CD5-APC and analysed by flow cytometry. The percentage of specific lysis in Cell-TrackerCD5⁺CD3⁻ CLL cells was determined by incorporation of 7-AAD (BD Pharmingen) and calculated according to the following formula: % Specific Lysis=100×(ER−SR)/(MR−SR). ER, SR, and MR represent experimental, spontaneous and maximum cell death.

### Statistical Analysis

For statistics, the following steps were followed:
1) Homogeneity was tested with KS and/or Shapiro-Wilk and/or D'Agostino & Pearson normality tests.
2) Bartlett test was used to check homogeneity of variance.
3) For parametric variables, ANOVA (one-way analysis of variance, followed by Dunett's multiple comparison test) or two-sample t-test were used to compare group means.
4) For non-parametric variables, Kruskal-Wallis (followed by Dunns multiple comparison test) and two-sample Mann-Whitney U tests were used to compare group medians. When paired samples were analysed, then the Wilcoxon or Friedman tests were used.
5) All data were analysed with Graph-Pad Prism 5 (GraphPad Software, San Diego, Calif.). All tests were two-sided. P<0.05 was considered to be statistically significant. Except otherwise stated, the mean and standard error of the mean (SEM) are provided for each group.

For the calculation of EC50 values, GraphPad Prism 5.0 (GraphPad Software, Inc.) was used. To this end, global non-linear regression, dose-response stimulation equation with a robust fit mode (standard slope) was selected.

### Results

Surface expression of CCR7 is maintained in CLL patients treated with BTK or Bcl-2 inhibitors.

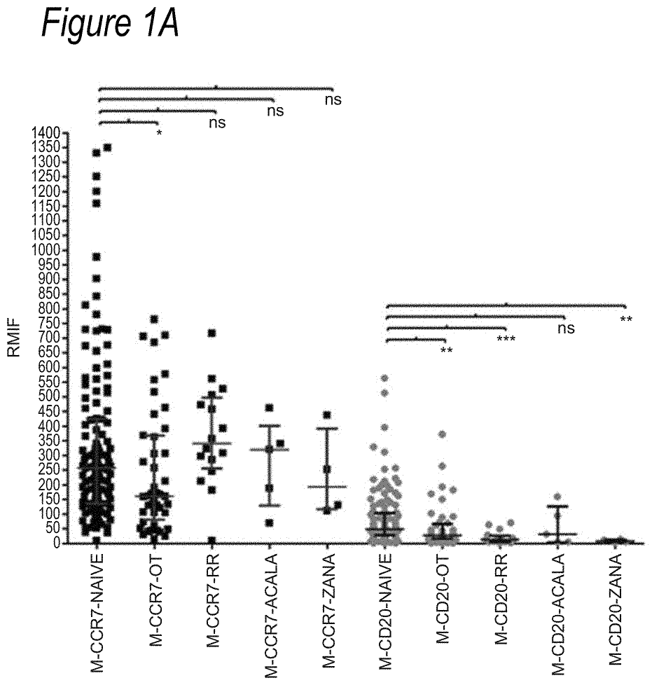

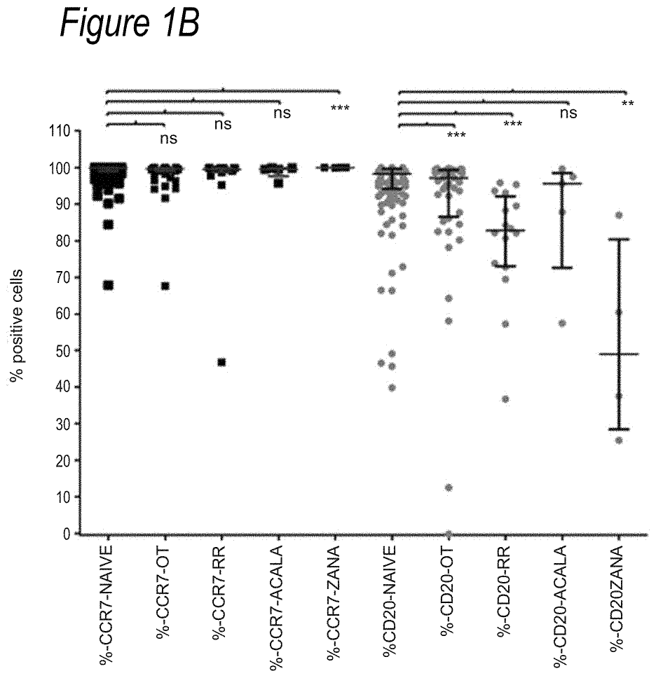

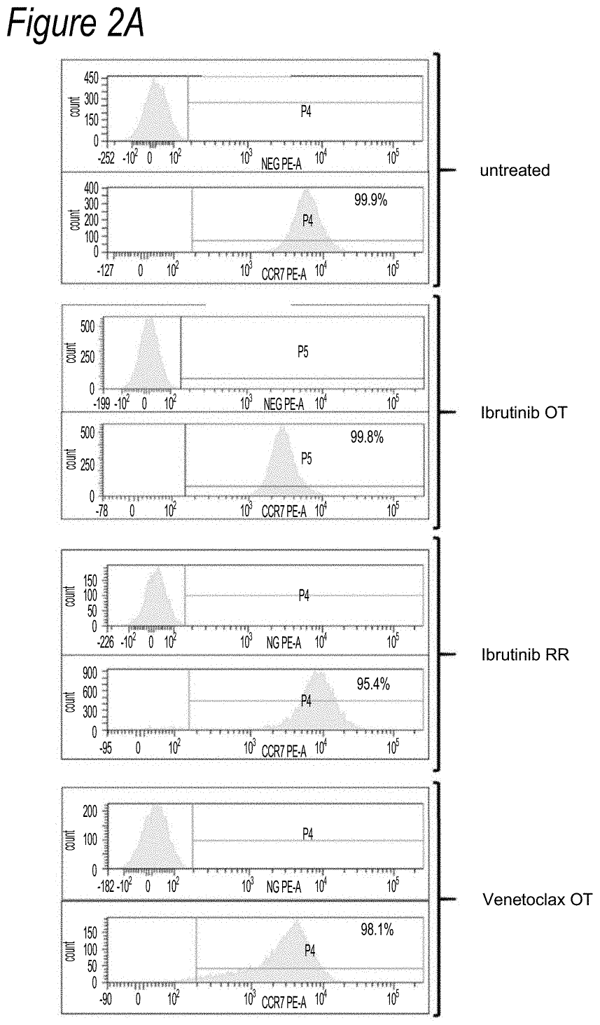

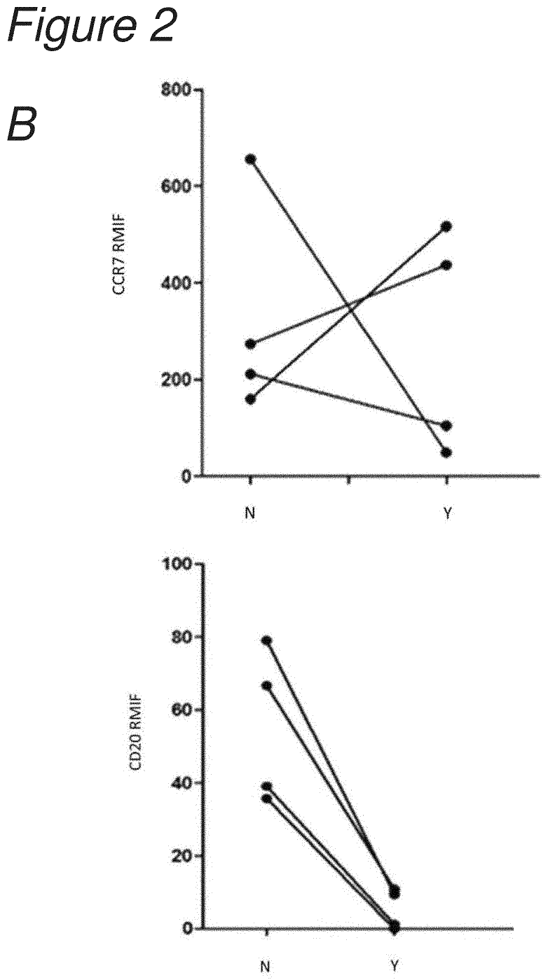

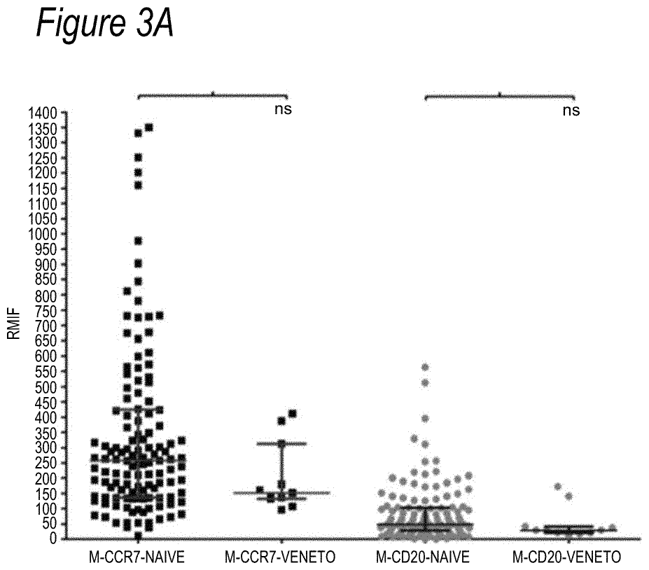

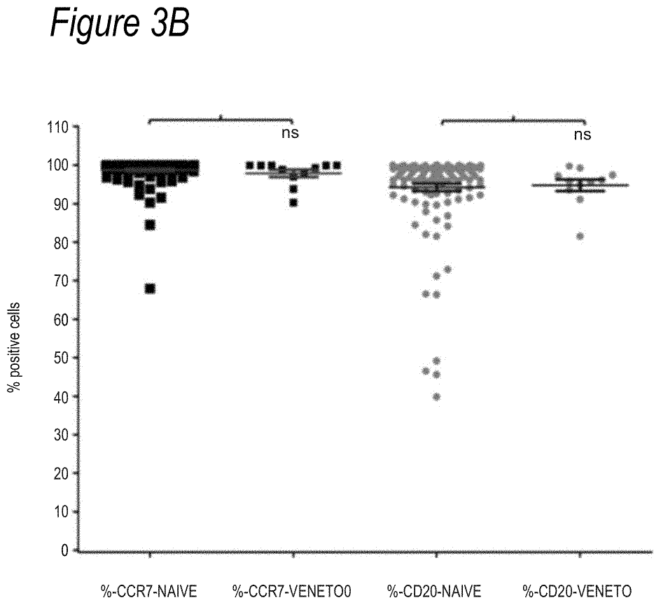

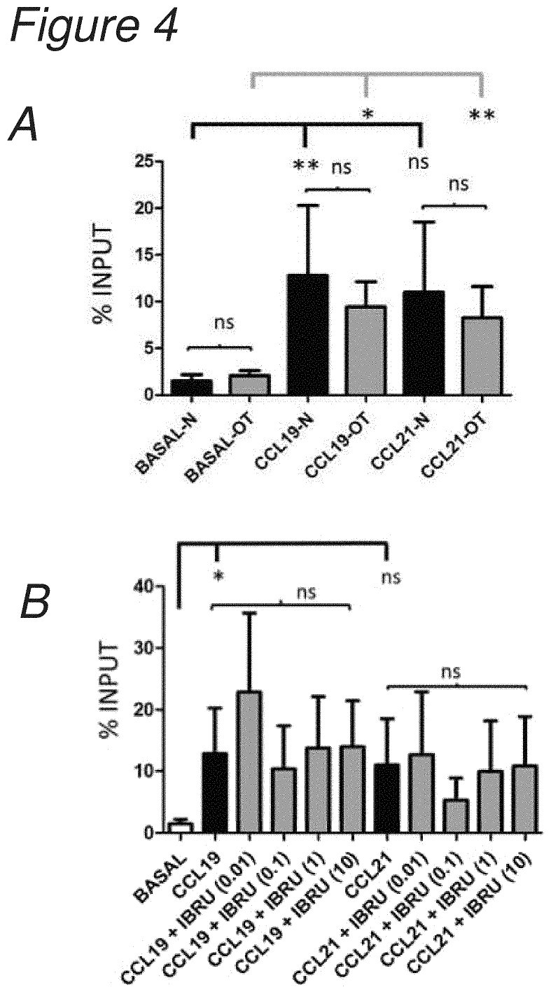

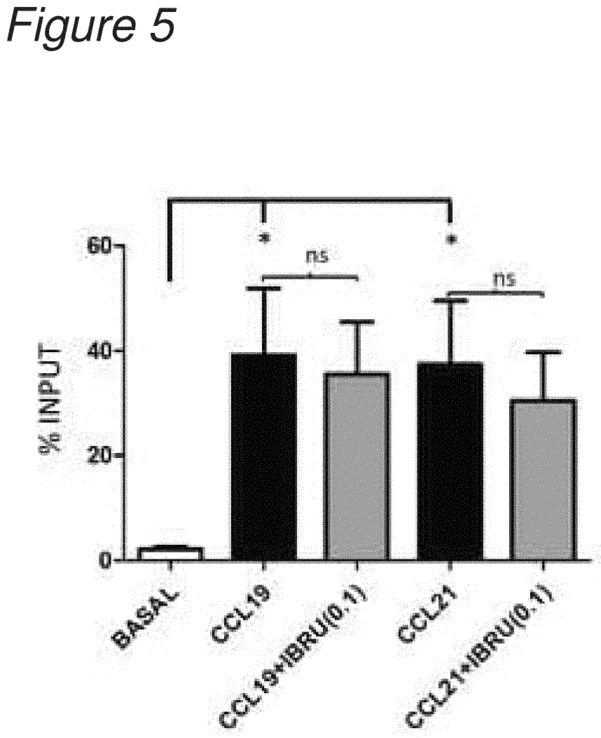

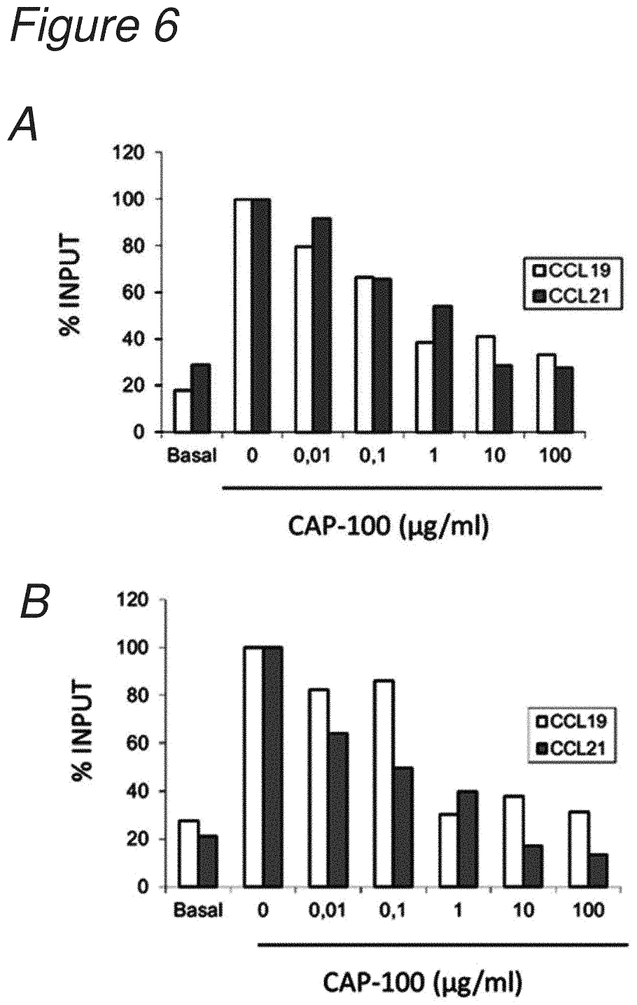

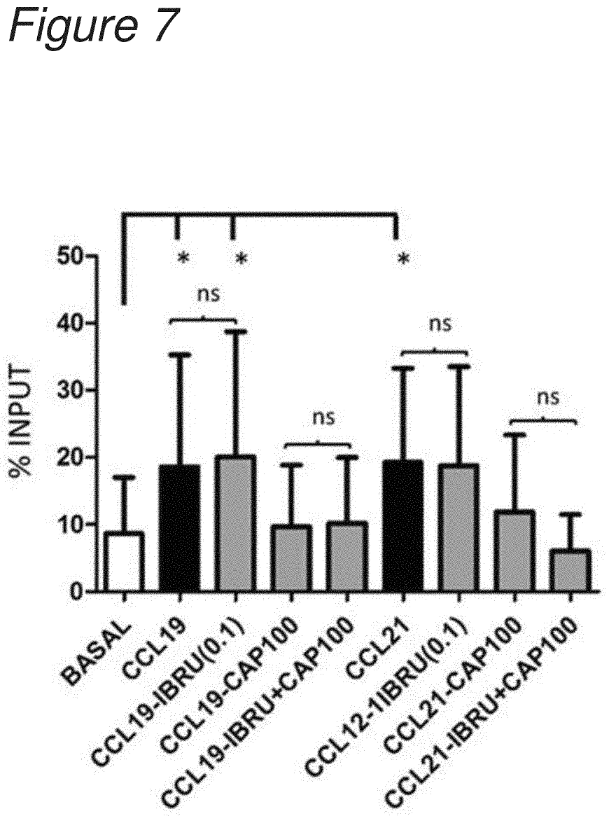

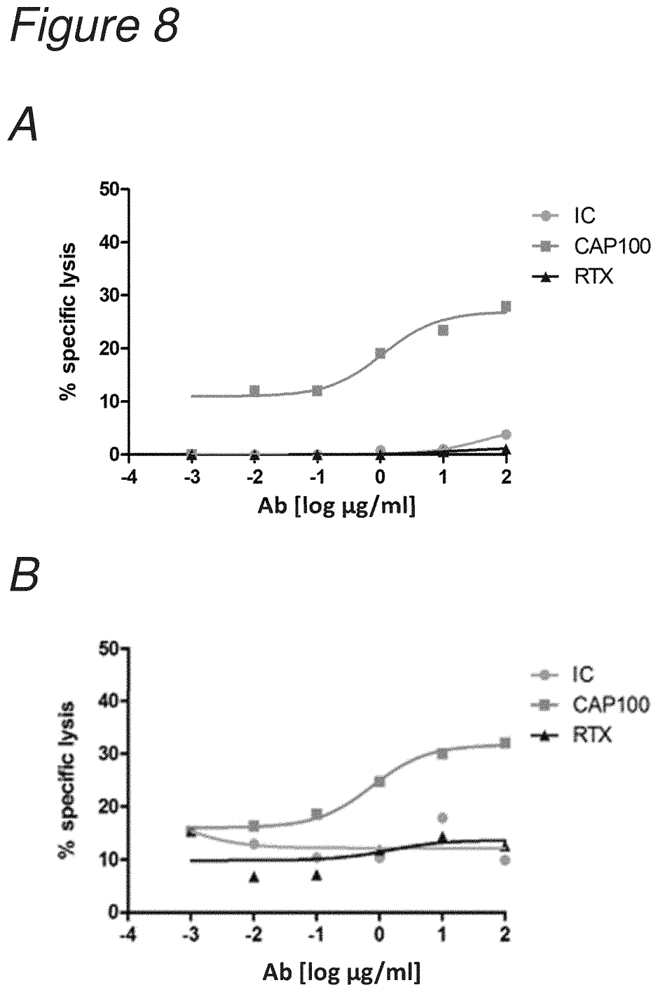

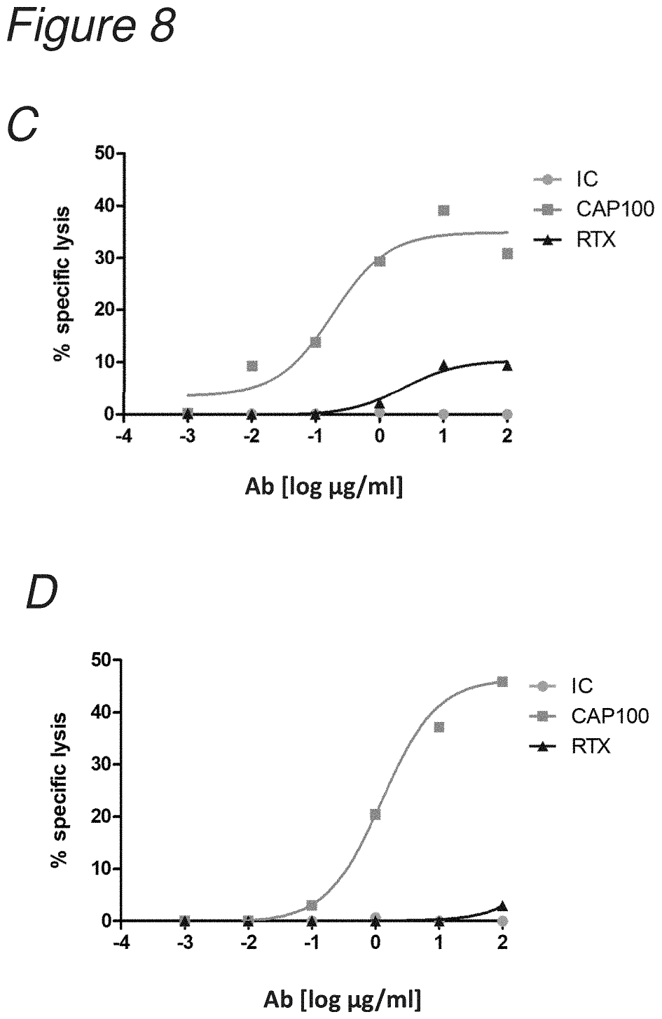

As previous works reported a substantial loss of surface CCR7 in CLL cells treated with ibrutinib, the first aim of the present study was to validate these results in a larger cohort of CLL patients. For this reason we measured surface CCR7 in 125 samples obtained from naïve patients (never treated before with either a BTK inhibitor or a Bcl-2 inhibitor), in 44 samples obtained from patients on ibrutinib treatment at the time of the determination, and in 16 samples obtained from patients who left ibrutinib treatment because they had developed relapsed/refractory disease. Strikingly, we observed that CCR7 expression was maintained during treatment with BTK inhibitors. As seen in FIG. 1-A-B and FIG. 2-A, we observed that patients receiving ibrutinib as current treatment, continued to show marked surface CCR7 expression. In general, a slight but significant down-modulation was observed in patients treated with ibrutinib, nonetheless approximately 100% of CLL cells maintained surface expression (as determined as percentage of CCR7-expressing cells), and also keep high surface levels (as determined as RMIF). Indeed, surface levels in treated patients were still over 200 arbitrary units. In other words, expression was still 200 times higher than a negative irrelevant isotype control. In patients on treatment with other BTK inhibitors such as acalabrutinib or zanabrutinib, no changes in CCR7 cells surface expression were observed. Finally, in the case of ibrutinib R/R CLL cells, CCR7 surface levels were similar to (or even higher than) those of naïve patients (FIG. 1-A-B and FIG. 2-A).

Interestingly, in the case of CD20, a receptor known to be down-modulated or lost by ibrutinib treatment, we confirmed that BTK-inhibitors led to a substantial down-regulation of CD20 surface levels. For example, treatment led to a complete loss of CD20 in some patients as determined by the reduction in the proportion of CD20-positive cells compared to naïve patients (FIG. 1-A-B). Accordingly, in FIG. 2-B is shown how ibrutinib treatment led to a consistent down-regulation of CD20 surface levels in four (4/4) patients as compared to the time they started ibrutinib treatment (Y), while in the case of CCR7 the overall picture remained essentially constant, with a strong reduction in one patient (1/4), a mild reduction in another patient (1/4), but increases in two other patients (2/4). Finally, and strikingly different from CCR7, CD20 expression in ibrutinib R/R CLL cells was even lower than in patients receiving ibrutinib (FIG. 1A and 2B).

Surface Expression of CCR7 is Maintained in CLL Patients Ttreated with Bcl-2 Inhibitors.

In CLL cells obtained from patients under treatment with venetoclax, we observed that approximately 100% of CLL cells maintained surface expression (as determined as percentage of CCR7-expressing cells), and also keep high surface levels (as determined as RMIF). As with ibrutinib, surface levels in venetoclax-treated patients were still about 150 - 250 times higher than a negative irrelevant isotype control (FIG. 2-A and FIG. 3-A-B.

Treatment with BTK Inhibitors Does Not Neutralize Migration Mediated by CCR7

Once it was determined that BTK inhibitors did not induce a meaningful loss of CCR7 on CLL cells surface we aimed to confirm that these compounds could have a negative impact on functions mediated by CCR7 through additional mechanism that could impair migration induced by CCR7 ligands. To this end, we performed a comparative analysis of the in vitro chemotactic response of CLL cells obtained from naïve patients (N) versus patients treated with ibrutinib (OT) (FIG. 4-A). In both groups of patients, basal migration indices were similar and, in both groups, CLL cells migrated in a significant way towards CCR7 ligands. Although a slight reduction was observed in the migration indices of the OT group, these values did not significantly differ from the N group, indicating that BTK-inhibitors had only a marginal effect on CCR7 induced migration. Moreover, migration indices in the OT group did not reach basal levels indicating that BTK inhibitor treatment does not completely neutralize migration induced by CCR7.

To further confirm this, we performed in vitro chemotaxis assays in transwell chambers. In these settings, CLL cells from naïve patients were incubated for one hour with increasing concentrations of ibrutinib (0; 0.01; 0.1; 1; 10 μM) before exposure to CCR7 ligands (black bars). As seen in FIG. 4-B, migration indices with ibrutinib pretreatment were comparable to control migration induced by CCR7 ligands. Only when administrated at 0.1 μM, ibrutinib showed a moderate (though not significant) effect on migration towards CCL21.

As the lack of ibrutinib inhibition could be associated to the short incubation times used (1 h prior to the chemotaxis+4 additional h during the chemotaxis assay) we decided to conduct new experiments where naïve CLL cells were incubated with ibrutinib at 0.1 μM for 24 h before testing chemotaxis. Again, as depicted in FIG. 5, no effect of ibrutinib was seen in CCR7-mediated migration of CLL cells towards CCL19, and a slight, not significant effect was observed in migration indices towards CCL21.

All together, these results indicated that CLL cells migration induced by CCR7 was not significantly affected by previous in vivo or in vitro treatments with ibrutinib. In other words, effects mediated by BTK inhibitors proved to be insufficient to abrogate CCR7-mediated migration, and agents blocking CCR7 are necessary to achieve a complete neutralization of CCR7-mediated migration to LNs. Nonetheless, the utility of anti-CCR7 as a blocking agent in patients receiving ibrutinib remained unaddressed. Moreover, as seen in FIG. 1-A, it was likely that the slight down-modulation of CCR7 surface levels could have a negative impact on the activity of CAP-100 in this group of patients on ibrutinib treatment. For these reasons we tested the neutralizing activity of CAP-100 in CLL cells obtained from 10 CLL patients treated with ibrutinib. As seen in FIG. 6, CAP-100 displayed a clear dose-response inhibitory activity on CLL cells obtained from one patient receiving venetoclax treatment (FIG. 6-A) or from one patient who failed to ibrutinib treatment (FIG. 6-B). In both cases, migration indices towards CCL19 or CCL21 reached basal levels after treatment with 10 or 100 μg/ml of antibody. These results confirmed that CLL cells from these groups of patients still respond to CCR7 ligands and that anti-CCR7 therapy probably is the best way to impair migration induced by CCR7.

We further tested whether combination of ibrutinib with an anti-CCR7 antibody such as CAP-100 could have an additive or synergistic neutralizing effect on the migration of CLL cells induced by CCR7 ligands. To this end, CLL cells from naïve patients were incubated with ibrutinib as single agent (0.1 μM), with CAP-100 as single agent (10 μg/ml), and with a combination of both compounds before exposure to CCL19 or CCL21. Selection of compound concentrations was based on previous findings on ibrutinib (FIG. 4B) and on CAP-100 (FIG. 6) as inhibitors of CCR7-mediated migration. As seen in FIG. 7, ibrutinib as a monotherapy had no effect on migration induced by CCR7, thus confirming results shown on FIG. 4B and FIG. 5. As expected, reduction of migration to basal levels was achieved with treatment with CAP-100, thus confirming that CAP-100 is a potent agent for inhibition of migration triggered by CCR7 ligands. When CCL21 was used as ligand, the combination of ibrutinib with CAP-100 showed a moderate, though not significant increase in the neutralizing activity obtained by anti-CCR7 antibody as single agent thus suggesting that a potential synergistic effect can be achieved by combining both compounds (FIG. 7). Although further validation is needed, this effect seemed to be specific for CCL21 as no similar outcome was observed when CLL cells treated with such a combination were exposed to CCL19.

CCR7 Expression in CLL Cells from Patients Under Treatment with BTK Or Bcl-2 Inhibitors can Effectively Induce Cell Death Upon Binding of an Anti-CCR7 Antibody.

In therapies based on antibodies, high target surface levels are mandatory to achieve an effective killing activity mediated by effector immune mechanisms such as ADCC, ADCP or CDC.

Indeed, proximity of two antibodies bound to target are needed to successfully complete any of the cited mechanisms [12]. Our results on expression demonstrated that surface CCR7 levels were slightly reduced after treatments with BTK inhibitors though no reduction on the proportion of tumour cells expressing the receptor was observed. For this reason, we determined whether the reduction in RMIF observed in patients treated with BTK inhibitors could have a negative impact on killing activities mediated by anti-CCR7 antibodies. To this end, we performed a set of ADCC assays, wherein target CLL cells from patients on ibrutinib treatment (FIG. 8A), on zanabrutinib treatment (FIG. 8B), on venetoclax treatment (FIG. 8C), or from ibrutinib R/R patients (FIG. 8D) were incubated with increasing concentrations of CAP-100, rituximab (used as reference therapeutic antibody), or an IC, and then, cells were incubated along with effector immune cells obtained from healthy donors at a fixed effector:target ratio of 10:1.

As seen in FIG. 8, CAP-100 demonstrated a potent ADCC activity in all four groups of patients. Remarkably, CAP-100 outperformed rituximab. These results confirm that, in the current settings, the slight reduction on surface CCR7 levels observed in patients treated with BTK or Bcl-2 inhibitors had no negative impact on immune effector mechanism mediated by antibodies such as ADCC. These results further confirm the utility of combinations based on BTK-inhibitors or Bcl-2 inhibitors with anti-CCR7 antibodies such as CAP-100.

1.-15. (canceled)

16. A method of treating a hyperproliferative hematological disorder, the method comprising administering an anti-CCR7 to a subject in need thereof, wherein the hyperproliferative hematological disorder is at least one of:
a) a disorder that is treated with at least one of a Bruton's tyrosine kinase (BTK) inhibitor and a B-cell lymphoma 2 (Bcl-2) inhibitor;
b) a disorder that has relapsed after treatment with at least one of a BTK inhibitor and a Bcl-2 inhibitor; and,
c) a disorder that is refractory to treatment with at least one of a BTK inhibitor and a Bcl-2 inhibitor.

17. The method of claim 16, wherein the antibody is administered simultaneously, separately or sequentially with at least one of a BTK inhibitor and a Bcl-2 inhibitor.

18. The method of claim 16, wherein the hyperproliferative hematological disorder is a disorder wherein the hyperproliferating cells are cells of the B cell lineage.

19. The method of claim 18, wherein the hematological malignancy is selected from the group consisting of acute lymphocytic leukemia (ALL), acute myeloid leukemia (AML), acute monocytic leukemia (AMoL), chronic lymphocytic leukemia (CLL), high-risk CLL, small lymphocytic lymphoma (SLL), high-risk SLL, multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), Burkitt's lymphoma (BL), hairy cell leukemia (HCL), Richter's transformation and T-cell prolymphocytic leukemia (T-PLL).

20. The method of claim 16, wherein the anti-CCR7 antibody has an IC₅₀ of no more than 100 nM for inhibiting at least one of CCR7-dependent intracellular signalling and CCR7 receptor internalization, by at least one CCR7-ligand selected from CCL19 and CCL21.

21. The method of claim 20, wherein the anti-CCR7 antibody inhibits CCR7-dependent intracellular signalling without substantial agonistic effects.

22. The method of claim 16, wherein the anti-CCR7 antibody has a K_{d} for the N-terminal extracellular domain of human CCR7 that is not more than a factor 20 higher than the K_{d} of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

23. The method of claim 16, wherein the anti-CCR7 antibody is a chimeric, humanized or human antibody.

24. The method of claim 23, wherein the anti-CCR7 antibody is an antibody having the HVRs of the anti-human CCR7 antibody of which the amino acid sequence of the heavy chain variable domain is SEQ ID NO: 1 and of which the amino acid sequence of the light chain variable domain is SEQ ID NO: 2.

25. The method of claim 16, wherein the BTK inhibitor is ibrutinib, zanabrutinib or acalabrutinib, and wherein the Bc1-2 inhibitor is venetoclax or navitoclax.

26. The method of claim 16, wherein the hyperproliferative hematological disorder is a disorder in a treatment-naïve patient.

27. The method of claim 16, wherein the hyperproliferative hematological disorder is a disorder in a patient who is naïve to the treatment with at least one of a BTK inhibitor, a Bc1-2 inhibitor and an anti-CCR7 antibody.

28. The method of claim 27, wherein the hyperproliferative hematological disorder is refractory to and/or has relapsed after treatment with a chemotherapeutic agent other than a BTK inhibitor, a Bcl-2 inhibitor and an anti-CCR7 antibody.

29. The method of claim 16, wherein the hyperproliferative hematological disorder is refractory to and/or has relapsed after treatment with at least one of a BTK inhibitor and a Bcl-2 inhibitor and a chemotherapeutic agent other than a BTK inhibitor, a Bcl-2 inhibitor and an anti-CCR7 antibody.

30. The method of claim 28, wherein the chemotherapeutic agent is one or more of fludarabine, cyclophosphamide, idelalisib, an anti-CD20 antibody.

31. The method of claim 30, wherein the anti-CD20 antibody is rituximab, obinituzumab, ocrelizumab, veltuzumab or ofatumumab, or an anti-CD52 antibody, wherein preferably the anti-CD52 antibody is alemtuzumab.


SEQUENCE LISTING (12 sequences)

SEQ ID NO: 1 - protein, 125 aa, Mus musculus
EVQLVESGGDLVKPGGSLKLSCVVPGLTFRDFAMSWVRQIPEKRLQWVATISSGGFYTYYPDSVKGRFTISRDNAKNTLYLQMNSLRSEDTAMYYCVRRAYRYDGTGDYSALDYWGQGTSVTVSS

SEQ ID NO: 2 - protein, 107 aa, Mus musculus
DIQMTQSPSSLSASLGERVSLTCRASQDIGPSLNWLQQGPDGTIKRLIYATSNLDSGVPKRFSGSRSGSDYSLTISSLESEDFVDYYCLQFASSPLTFGAGTKLELK

SEQ ID NO: 3 - protein, 32 aa, Artificial
  SYM1899 synthetic sulfated peptide corresponding to amino acids 19-49 of human CCR7
  MOD_RES (1)..(1): PYRROLIDONE CARBOXYLIC ACID
  MOD_RES (8)..(8): SULFATATION
  MOD_RES (17)..(17): SULFATATION
EDEVTDDYIGDNTTVDYTLFESLCSKKDVRNK

SEQ ID NO: 4 - protein, 125 aa, Artificial
  Humanized VH1 domain
EVQLLESGGGFVKPGGSLKLSCVVSGLTFRDFAMSWVRQTPEKRLVWVATISSGGFYTYYPDSVKGRFTISRDNVRNILYLQMSSLRSEDTAVYYCVRRAYRYDGTGDYSALDYWGTGTTVTVSS

SEQ ID NO: 5 - protein, 107 aa, Artificial
  Humanized VK1 domain
DIQMTQSPSSLSASVGDRVTITCRASQDIGPSLNWYQQKPGKAPKRLIYATSNLDSGVPSRFSGSGSGTDFTLTISSLQPEDFATYYCLQFASSPLTFGQGTKVEIK

SEQ ID NO: 6 - protein, 107 aa, Artificial Sequence
  Synthetic human antibody
SYELTQPPSVSVSPGQRARITCSGDKLGDKYASWYQQKPGQSPLLVIYQDSKRPSGIPERFSGSNSGNTATLTISGTQAMDEADYYCQAWGSSTVIFGGGTKLTVLG

SEQ ID NO: 7 - protein, 106 aa, Artificial Sequence
  Synthetic human antibody
YELTQPPSVSVSPGQRARITCSGDKLGDKYASWYQQKPGQSPLLVIYQDSKRPSGIPERFSGSNSGNTATLTISGTQAMDEADYYCQAWGSSTVIFGGGTKLTVLG

SEQ ID NO: 8 - protein, 124 aa, Artificial Sequence
  Synthetic human antibody
QVQLVESGGGVVQPGRSLRLSCAASGFTFSSYGMHWVRQAPGKGLEWVAVIWYDGSKKYYADSVKGRFTISRDNSKNTLYLQMNRLRAEDTAVYYCARDGGIAVFLQDNWFDPWGQGTLVTVSS

SEQ ID NO: 9 - protein, 330 aa, Homo sapiens [sequence omitted]

SEQ ID NO: 10 - protein, 330 aa, Artificial
  synthetic heavy chain constant region of the human allotype G1m (17,1) with a E333A substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, Artificial Sequence
  Synthetic human antibody heavy chain
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTISYTPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Synthetic human antibody light chain
EIVLTQSPGTLSLSPGERATLSCRASQSVSSSYLAWYQQKPGQAPRLLIYGASSRATGIPDRFSGSGSGTDFTLTISRLEPEDFAVYYCQQSYSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC